(19) United States
(12) Patent Application Publication
(10) Pub. No.: US 2022/0412961 A1
(43) Pub. Date: Dec. 29, 2022

(54) DEVICE AND METHOD FOR ANALYTE DETECTION

(71) Applicants: PRESIDENT AND FELLOWS OF HARVARD COLLEGE, Cambridge, MA (US); MASSACHUSETTS INSTITUTE OF TECHNOLOGY, Cambridge, MA (US)

(72) Inventors: Pawan JOLLY, Boston, MA (US); Helena DE PUIG GUIXE, Barcelona (ES); Olivier Yves Frederic HENRY, Conconmam (FR); Donald E. INGBER, Boston, MA (US); James J. COLLINS, Newton, MA (US); Nur MUSTAFAOGLU, Boston, MA (US); Nooralhuda ARKAN, Beavercreek, OH (US)

(73) Assignees: PRESIDENT AND FELLOWS OF HARVARD COLLEGE, Cambridge, MA (US); MASSACHUSETTS INSTITUTE OF TECHNOLOGY, Cambridge, MA (US)

(21) Appl. No.: 17/776,687

(22) PCT Filed: Nov. 13, 2020
(86) PCT No.: PCT/US2020/060374

§ 371 Date: May 13, 2022

Related U.S. Application Data
(60) Provisional application No. 62/936,038, filed on Nov. 15, 2019.

(51) Int. Cl. G01N 33/52; G01N 33/543; C07K 16/44

(52) U.S. Cl. CPC G01N 33/525; G01N 33/54353; G01N 33/54393; C07K 16/44

(57) ABSTRACT

Embodiments of various aspects described herein are directed to assays and devices for detecting a target molecule in a sample. In particular, there is described a lateral assay comprising a plurality of serially oriented capture zones, where each capture zone independently comprises an immobilized competitive molecule on a lateral flow matrix. The immobilized competitive molecule and the analyte competitively bind with a capture agent capable of binding the analyte.

### CROSS-REFERENCE TO RELATED APPLICATIONS

This application claims benefit under 35 U.S.C. § 119(e) of U.S. Provisional Application No. 62/936,038 filed Nov. 15, 2019, the content of which is incorporated herein by reference in its entirety.

### TECHNICAL FIELD

The description herein relates generally to methods, compositions, and kits for detecting a target entity in a sample. In particular methods and compositions for detecting small molecules using a lateral flow assay are described.

### BACKGROUND

Detection of small molecules (<1000 Da) is a challenging field because it is difficult for an antibody to recognize and bind to a small number of functional groups. These antibodies are typically raised by conjugating the small molecule target to a larger protein carrier followed by inoculation of the animal. Such an approach has been found to be successful for a number of targets that contain multiple functional groups (for example cortisol, testosterone). However, very small molecules, such as histamine (111.14 g/mol), which consists of an imidazole ring and a short carbon chain terminated with a primary amine, still pose a challenge due in part to their limited functionalities. This is an important challenge because antibody-based diagnostics that can detect small molecules such as histamine with high specificity and sensitivity have great potential value for medical diagnostics (e.g., for allergy and anaphylaxis), early detection of diseases, and food safety applications.

Antibodies against small molecules such as histamine are typically raised by conjugating the small molecule to a large immunogenic protein carrier, such as bovine serum albumin (BSA) or ovalbumin (OVA). Consequently, only a portion of the small molecules will be exposed to the lymphocytes, which commonly results in the generation of antibodies that specifically recognizes the protein-bound small molecule and not the free floating small molecule. For example, in the case of histamine, only the imidazole will be exposed to lymphocytes, which results in generation of protein-bound histamine specific antibodies having only limited affinity and sensitivity for free histamine. These antibodies typically perform poorly in the development of immunoassays for the target small molecule released in a free form from tissues, which is often most clinically relevant. Thus, there is a need to design ways to overcome this lack of specificity of currently available antibodies targeted to small molecules.

Most of the studies on histamine detection are based on the use of either protein-conjugated histamine molecule bound via its primary amine group, or chemically modified histamine, both for antibody development and as specific competitive inhibitors of binding in immunoassays. For instance, Morel et al. developed antibodies using chemical derivatization where an acylating reagent was synthesized to raise monoclonal antibodies against acylated histamine [Morel, A. M. and Delaage, M. A., 1988, “Immunoanalysis of histamine through a novel chemical derivatization,” Journal of allergy and clinical immunology, 82(4), pp. 646-654]. As a result, the antibodies produced showed greater affinity towards the derivatized histamine than free histamine. Buckler et al. describe various type of histamine derivatives for antibody production [Buckler, et al., U.S. Pat. No. 5,112,738]. Nearly all of the haptens presented in this publication involves different ways to attach the histamine molecule (either from the carbon tail or the imidazole ring) to a carrier or a terminal functional group. The study showed that a hapten produced by conjugating histamine to a protein carrier was the most efficient way to produce monoclonal antibodies against histamine. More recently, Mattsson et al. presented a detailed study on the development of a histamine assay using commercial antibodies [Mattsson, L., Doppler, S. and Preininger, C., 2017, “Challenges in Developing a Biochip for Intact Histamine Using Commercial Antibodies,” Chemosensors, 5(4), p. 33.]. The research group tested six commercial antibodies out of which only two showed affinities towards free histamine. However, even these two antibodies demonstrated poor sensitivity in the μg/mL range for the histamine molecule.

There are a variety of techniques that have been used to detect histamine which include high performance liquid chromatography (HPLC), gas chromatography (GC), molecularly imprinted polymers (MIP) and enzyme linked immune sorbent assay (ELISA). However, there has not been a reported cost-effective and rapid method of detecting histamine with the range of [5 nM-100 nM] in blood or other matrices [Marloes P. et al., Impedimetric Detection of Histamine in Bowel Fluids Using Synthetic Receptors with pH-Optimized Binding Characteristics. Anal. Chem. 2013, 85, 3, 1475-1483]. The speed of detection is particularly important as anaphylactic shock can result with minutes after early symptoms of allergic reaction are first detected. Thus, a histamine-based anaphylaxis diagnostic would need to be able to detect rises in histamine levels within less than 20 minutes after the first symptoms being detected, and preferably within 5-10 minutes.

In the field of low-cost diagnostics, paper-based strategies have gained a huge interest. For instance, the lateral flow assay (LFA) is a low cost point-of-care (POC) diagnostic that involves simple fabrication and enables assays to be completed in a short time (minutes). Because of the small molecular weight of histamine, competitive lateral flow assays are required for its detection. Typically, competitive immunoassays for small molecule detection use BSA conjugated to the molecule of interest. BSA-small molecule conjugates are immobilized on nitrocellulose and small molecule-binding antibodies are attached to gold nanoparticles. The antibodies on the nanoparticles recognize the BSA conjugates and bind to them, forming a red spot as a result of gold nanoparticle accumulation. If the small molecule is present in the solution, it competes for the antibody binding sites and blocks the antibody from binding to the small molecule on the BSA; resulting in a lower signal. Increasing concentrations of the small molecule lead to decreased signals in the immunoassay.

Currently, there are two LFA tests for histamine that are commercially available. REVEAL® from Neogen which a 5-minute test for histamine is detection and HISTASURE™ from Labor Diagnostika Nord GmbH & Co. KG which is a 10-minute test. Both tests are used for the detection of histamine in fish products because this is an indicator of food spoilage. The tests also require an acylation step of samples before running them on the strip together with sample preparation. These commercial assays have a sensitivity of 50 ppm (450 μM), which is 4-5 orders of magnitude higher than the histamine concentrations in blood (5-100 nM).

Therefore, in addition to low sensitivity and selectivity, the reported methods for detection of small molecules are inefficient, often requiring incubation times of more than an hour to evaluate the presence of small molecules. Hence, there remains a need for the development of more sensitive immunoassays and surpass the limitations of currently available inefficient low affinity small molecule antibodies.

### SUMMARY

Embodiments of various aspects described herein, include development of a device and assay for detection of small molecules. For example, the device can be in the form of a lateral flow assay for detection of small molecules such as histamine in a biological sample, such as from a test subject.

In one aspect the disclosure provides a lateral flow assay device for detecting the presence of a small molecule analyte in a liquid sample, comprising a lateral flow matrix which defines a flow path and which comprises in series: a sample receiving zone; and one or more capture zones. In this device, each capture zone independently comprises a conjugate immobilized on the lateral flow matrix, wherein the conjugate comprises one or more analyte-related molecules conjugated to the lateral flow matrix via a linker and wherein the linker is not a protein. The conjugate is adapted for orienting the analyte-related molecule for binding with a capture agent capable of binding specifically with the analyte, wherein the analyte-related molecule and the analyte competitively bind with said capture agent.

Optionally, the analyte is selected from the group consisting of amino acids, nucleosides, saccharides, steroids, hormones, therapeutic agents, metabolites of therapeutic agents. Optionally, the analyte-related molecule comprises an imidazole group, for example, wherein the analyte-related molecule is histadine. Optionally, a binding affinity of the analyte binding with the capture agent is higher than a binding affinity of the immobilized conjugate binding with the capture agent.

Optionally, the analyte is histamine.

In some options, the linker has a length between 5 and 200 angstroms. Optionally, the linker comprises a polyethylene glycol (PEG). Optionally, the linker comprises at least one lysine, wherein, optionally at least one analyte-related molecule is linked to the alpha-amino group of the at least one lysine and at least one analyte-related molecule is linked to the epsilon-amino group of the at least one lysine. Optionally, the linker comprises a first lysine linked to a second lysine, and wherein the carboxyl group of the first lysine is linked to the epsilon-amino group of second lysine. Optionally, the linker comprises a first lysine, a second lysine and a third lysine, and wherein the carboxyl group of the first lysine is linked to the epsilon-amino group of the second lysine, and the carboxyl group of the third lysine is linked to the alpha-amino group of the first or second lysine.

Optionally, the capture agent is an antibody. Optionally, the capture agent comprises a detectable label.

In some options, the device comprises a plurality of serially oriented capture zones. Optionally an amount of the immobilized conjugate in at least two capture zones is different. Optionally, an amount of the immobilized conjugate in a capture zone closer to the sample receiving zone is lower than an amount of the immobilized conjugate in a capture zone further from the sample receiving zone. Optionally an amount of the immobilized ligand in each capture zone is lower than an amount of the immobilized ligand in each capture zone that is further from the sample receiving zone.

Optionally the device further comprises a first control zone, wherein the first control zone comprises an analyte molecule immobilized on the lateral flow matrix. Optionally, the first control zone comprises a BSA conjugated to the analyte molecule. Optionally, the analyte molecule is histamine. Optionally, the first control zone is positioned next to a capture zone so that the distance from the sample receiving zone to the control zone and the distance from the sample receiving zone to the capture zone are substantially equal. Optionally, the device further comprises a second control zone comprising an anti-Fc capture agent. Optionally this second control zone is positioned in series after the capture zones, wherein a liquid flowing from the sample zone reaches the capture zones before reaching the second control zone.

Optionally, the sample receiving zone comprises: (i) a labeling zone comprising a diffusively bound capture agent; and (ii) a sample zone for receiving a liquid sample comprising the analyte. Optionally, the labeling zone is positioned between the plurality of capture zones and the sample zone for receiving the liquid sample comprising the analyte.

Optionally, each capture zone independently has a regular or irregular shape. For example, optionally at least one of the capture zones has a shape selected from the group consisting of a line, a circle, a rod, and a polygonal. In some options the polygonal is a square, a triangle or a rectangle. In some embodiments, at least two captures zones are of same shape. In some embodiments, at least two captures zones are the same size.

Another aspect provided herein relates to a method for detecting the presence of an analyte in a liquid sample, the method comprising: (i) contacting the liquid sample to the sample receiving zone of the device according to the first aspect, wherein the capture agent comprises a detectable label; and (ii) observing a detectable signal from the detectable label in the capture zones, wherein the detectable signal is inversely proportional to a concentration of the analyte in the sample.

In some embodiments, the method further comprises combining the detectable signals from two or more capture zones to provide a processed signal. Optionally, the detectable signal is provided as a quantified value and combining comprises summing or averaging the detectable signals from said two or more capture zones to provide the processed signal. Optionally wherein said combining the detectable signals comprises averaging the detectable signal.

The devices and methods as described herein provide low sensitivity and high selectivity for the detection of small molecules such as histamine. For example, efficient assays are provided that can detect less than 450 μM of targeted analytes in less than 10 min.

### BRIEF DESCRIPTION OF THE DRAWINGS

This patent or application file contains at least one drawing executed in color. Copies of this patent or patent application publication with color drawings will be provided by the Office upon request and payment of the necessary fee.

FIG. 1 shows an embodiment of an immunochromatograph test strip.

FIG. 2 shows another embodiment of an immunochromatograph test strip.

FIGS. 3A and 3B are bar graphs for the results from a half strip assay test using single BSA-histamine/BSA-Histidine spot on the membrane. FIG. 3A 0.5 mg/ml BSA spot, FIG. 3B and 0.1 mg/ml BSA spot.

FIG. 4 is a plot showing the effect of single and multiple spots in an LFA analysis for histamine.

FIG. 5A shows a calibration curve.

FIG. 5B are images of exemplary LFA strips used to make the calibration curve shown in FIG. 5A.

FIG. 6 shows a LFA according to some embodiments used for detecting 0, 50, 100 and 5000 nm of histamine.

### DETAILED DESCRIPTION OF THE INVENTION

Embodiments of various aspects described herein relate to methods, compositions and kits for detecting small molecules. The inventors have discovered inter alia that a multivalent test spot and multiple test spots provide an efficient (e.g., sensitive and rapid) detection of small molecules, such as histamine.

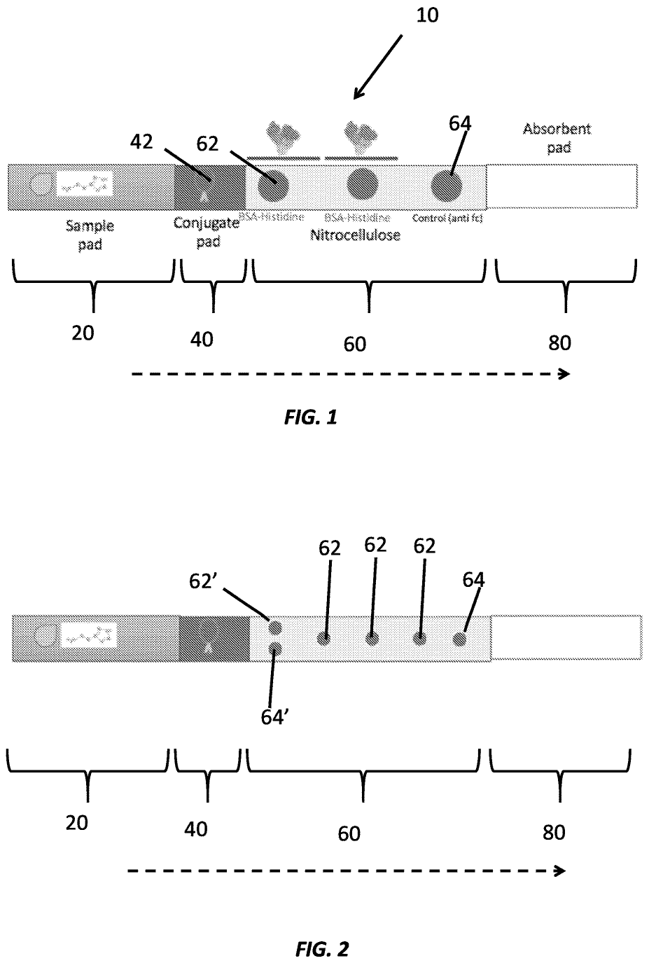

FIG. 1 shows an embodiment according to some aspects of the invention and depicts a top down view of an immunochromatograph test strip, also known as lateral flow immunoassay (LFA). The test strip, 10 includes a lateral flow matrix that can include several sections, pads or components in series that are connected and configured to be in fluid communication with each other. Under normal operation, the primary flow direction of a fluid containing the sample is as indicated by the dashed arrow, passing from the sample receiving zone 20, through the optional labeling zone 40, through the testing zone 60, and to the absorbent pad 80. The LFA is designed so that in each of the sections 20, 40, 60, and 80 the fluid carries the sample through the sections via capillary action and the sample can interact with sub components in the LFA sections.

In some embodiments the LFA is configured as a competitive assay. This kind of assay relies on establishing an equilibrium between a target analyte of interest, a capture agent capable of specifically binding to the analyte, and a conjugate immobilized on the LFA thereby providing an immobilized conjugate. The attachment of the capture agent to the conjugate immobilized on the LFA is detected so that where the concentration of the target analyte in increased, the capture agent is displaced or does not attach to the capture zone. Therefore, in this kind of assay, the detectable signal is inversely proportional to the concentration of the analyte.

In some embodiments, the testing zone or region 60 includes a plurality of capture zones 62, two of which are shown in FIG. 1. The capture zones 62 include the conjugate that are immobilized or attached to the surface of the LFA. In some embodiments, the concentration of the conjugate in at least two of the plurality of capture zones is different. In some embodiments region 60 also includes control zones, such as control zone 64. The labeling zone section 40 can include one or more conjugation-zone 42. The conjugation-zone 42 includes diffusively bound capture agent that can bind to both an analyte in the sample, when present, and to the conjugate in capture zones 62. The term “diffusively bound” means loosely held so that the capture agent can be carried with a fluid, such as the fluid provided with a sample. For example, the capture agent can be bound to the analyte and can be carried by the fluid. The capture agent, released from the conjugation-zone 42, is carried with a fluid flowing through test strip 10 along with the sample and interacts with the capture zones 62, and the optional control zone 64. Although depicted as a single zone, there can be a plurality of capture zones 62 and/or conjugation-zones 42 according to some embodiments.

In the LFA, the components can be mounted on a backing material, such as a card having a pressure sensitive adhesive. The backing can provide structural support. The components can all comprise a single material such as a sheet or web of nitrocellulose, PVDF, polyethylene, nylon, cellulose acetate, polyester, polyethersulfone or polysulfone. Alternatively, one or more of the components can be made of different materials. The sheet and webbing material can be a porous material. For example, the material can have an interconnected porosity so that the materials can wick and flow fluid through them with a constant flow rate. As used herein “webbing” or “web” indicates a flexible material that can be made from polymers or fibrous materials such as a woven or non-woven textile, paper or felt.

Some embodiments of the test strip 10 include only a subset of the sections. For example, in a simplified version only the testing zone 60 is included, such as in a dot or blot test. In the dot or blot configuration the strip is entirely contacted (e.g., immersed or covered with) the sample which includes the target analyte and conjugate in a liquid.

In another embodiment, referred to as a half stick assay, the absorbent pad 80 and testing zone 60 are included. In the half stick assay, the end distal from the absorbent pad 80 can be contacted with sample, which includes the target analyte and capture agent, for example by placing the end into a reservoir of the liquid containing the sample so that the capture zones 62 are not immersed in liquid. By this method, the end of the test zone 60 distal from the absorbent pad 80 acts as a sample zone.

The absorbent pad 80, also known as a wick or waste reservoir, is designed to pull at least a part of, e.g., all the fluid added to the strip into this region and to hold it for the duration of the assay. Thus the absorbent pad 80, in normal operation, causes the material to flow from 20 to 80, e.g., in the direction indicated by the dashed arrow. In addition to the sheet and webbing materials previously mentioned, the wick can be chosen to have a high absorptive capacity such as a high-density cellulose (e.g., chromatograph paper).

In another embodiment the sample receiving zone 20 is included. If the labeling zone 40 is not included in this configuration, the sample receiving zone 20 is contacted on one end with the testing zone 60, and if a capture agent is needed, the capture agent can be included in the liquid containing the sample. When using the lateral flow assembly, the sample can be applied to the sample receiving zone 20, for example using an applicator (e.g., a pipette to drip sample on the sample receiving zone) or it can be dipped in the sample solution. In some embodiments the sample receiving zone 20 is simply an area for addition of the sample, in other embodiments the sample receiving zone 20 can function to modify the sample (e.g., to filter out particulate or cells, or modify the pH of the solution) before it flows to other portions of the device. In some embodiments the sample receiving zone 20 can include the sheet or web material previously described, or it can include cellulose, glass fiber, rayon and other filtration media. From the sample receiving zone 20, the analyte containing solution flows toward the testing zone 60.

In some embodiments, a conjugate pad 40 is used and is placed between the sample receiving zone 20 and testing zone 60. The conjugate pad 40 can be used to hold a capture agent needed in the assay. In some embodiments, the capture agent can be confined to a conjugation zone 42 in or on the conjugate pad 40. For example, a labeled capture agent, e.g., an antibody can be contained in the conjugate pad 40, such as in the conjugation zone 42, until it is contacted with the sample solution, wherein it mixes with the solution and can function as intended e.g., to bind to an analyte, the capture zones 62, or the control zones 64. In addition to the sheet or web material previously mentioned, the labeling zone 40 can comprise glass fibers, polyesters, or rayon. In some embodiments the sample receiving zone 20 and labeling zone 40 are combined. For example, the sample receiving zone can include a section that includes a conjugate zone 42.

The control zone 64 is an optional feature and is functionalized so that it will indicate that a sample has been applied to the assay. For example, the control zone 64 can be functionalized with a molecule that binds with the capture agent, e.g., a non-specific antibody. Although shown as a single zone, there can be a plurality of control zones 64. The control zones 64 can also be placed laterally in any position on the LFA, such as close to the sample receiving zone 20 e.g., before any of the capture zones 62, to any capture zone 62, between some of the capture zones 62 of after all of the capture zones as shown in FIGS. 1 and 2. “Next to” in this context refers to a positon in the direction perpendicular to the liquid flow (dashed arrow), such that a fluid containing the sample reaches a control zone 64 and a capture 62 at approximately the same time when they are next to each other. As shown in FIGS. 1 and 2, a fluid containing the sample reaches the control zone 64 after reaching the capture zones 62. In some embodiments the control zone 64 is functionalized with an anti-Fc capture agent. In these embodiments, the control 64 can pull down Ab coated beads that are in the fluid contacting 64.

In some embodiments, a control zone 64′ is place next to a capture zone 62 as depicted by FIG. 2, where control zone 64′ is next to a capture zone 62′ that is closest to the sample receiving zone 20. In such embodiments the distance from the sample receiving zone 20 to the control zone 64′ and the distance from the sample receiving zone 20 and the capture zone 62′ are approximately the same. Where different control zones are used, indicated as 64 and 64′ in FIG. 2, the composition of the control zones can be independently varied, for example, to control for a particular characteristic of the sample. In some embodiments a control zone 64′ can be next to any capture zone of a plurality of capture zones 62. In some other embodiments two or more control zones 64′ are used and placed next to any one, two or more capture zones 62, or placed in any position in the testing zone 60. In some embodiments the control zone 64′ provides a baseline signal such as a color intensity the indicates the state (e.g., related to any degradation thereof) of a conjugate in a competitive assay, such as an antibody or an antibody-nanoparticle conjugate. In some embodiments, the control zone signal 64′ is used for normalization of the signals from the detection zones 62′. In some embodiments the control zone 64′ includes an analyte molecule comprised in a conjugate and further linked to a carrier molecule, e.g., BSA. In some embodiments the analyte molecule is histamine.

Without being bound to any specific theory, the use of a first control 64′, where the first control is a BSA-histamine conjugate, use of a second control 64, where the second control includes an anti-Fc capture agent, and use of capture zones 62 including histidine, can be beneficial for assays directed to detection of histamine for the following reasons. The first control 64′ pulls down Ab coated beads and provides calibration for Ab activity as opposed to control 64 which indiscriminately detects Ab presense. Ab activity degrades more quickly than the Ab Fc region, which would in turn reduce the amount of signal from the sample. Therefore, the first control 64′ provides an indication of the quality of the Ab functionalized beads whereas the second control zone 64 provides an indication as to whether a solution with some Ab beads has flowed through the test strip and therefore the general operation and state of the test strip.

The LFA can be enclosed in a housing. The housing can be made of any useful material such as a rigid molded plastic. The housing can have openings and window access/viewing areas appropriately placed for operation of the device. For example, an opening for application of the sample on the sample receiving zone and openings or windows for viewing or analysis of the capture zones 62 and control zones 64.

In some embodiments the amount of conjugate in a capture zone 62 closer to the sample zone 20, or where the sample is introduced to the LFA, is lower that the amount of conjugate further from the sample introduction area 20. For example, the concentration can be at least 1% higher, at least 5% higher, at least 10% higher, at least 50% higher, at least 100% higher, or even 10 times or 100 times higher in the capture zone 62 further away from the sample zone 20. In some embodiments, where more than two capture zones 62 are used, there is a concentration gradient of increasing concentration of conjugate between each independent capture zone 62, where the gradient increases in the direction from the sample introduction zone 20 to the adsorbing pad 80. In some embodiments the concentration gradient is a constant gradient, so that the concentration of conjugate increases by the same amount between each sequential capture zone. In some embodiments, the concentration gradient in not constant and varies in magnitude between at least two sequential capture zones. For example, in some embodiments the concentration increase of conjugate between sequential capture zones are not constant but vary in a fixed manner such as by multiples of two, where in each sequential capture zone the conjugate are doubled, or by orders of magnitude, where each sequential capture zone has an order of magnitude higher concentration of the conjugate. In some embodiment the amount of conjugate in each of the capture zones is between about 0.01 ng and about 2 ng, between about 0.05 and about 1 ng, between about 0.1 and about 0.5 ng, or between about 0.125 ng and about 0.25 ng.

The conjugation-zone 42, capture zones 62, and control zones 64 and 64′, are shown with a circular shape in FIGS. 1 and 2, but they can independently have different shapes as viewed top down on the LFA. For example, each of conjugation-zone 42, capture zones 62, and control zones 64 and 64′, independently can have a regular or irregular shape. As used here, a “regular shape” refers to a shape having at least one symmetry element perpendicular to the plane of the LFA, such as at least one mirror plane or rotational axis perpendicular to the surface of the LFA. For example, a regular shape can include any of a line, a circle, an oval, a semicircle, a rod, an ellipse, and an n-sided regular or irregular polygon where n can be any integer greater than 3, such as between 3 and 100, and regular refers the sides being equal while irregular refers to one or more sides being un-equal. For example, the polygon can include a lozenge or diamond shape, a triangle, a square, a pentagon, a hexagon, an octagon, a star shape, a rectangle, or a parallelogram. In some embodiments the conjugation-zone 42, capture zones 62, and control zones 64 and 64′ all have the same shape. In some embodiments all of the capture zones 62 have the same shape.

In some embodiments the conjugation-zone 42, capture zones 62 and 62′, and control zones 64 and 64′, have an area between about 0.01 mm² and about 1 cm², between about 0.1 mm² and about 0.5 cm², between about 0.5 mm² and about 2 mm². In some embodiments each of the conjugation-zone 42, capture zones 62 and 62′, and control zones 64 and 64′ independently have a different size. In some embodiments at least two of the capture zones 62 have the same size.

As used herein, a “capture agent” means a molecule or composition capable of binding with the analyte. Exemplary capture agents include peptides; polypeptides; proteins; peptidomimetics; antibodies; antibody fragments (e.g., antigen binding fragments of antibodies); carbohydrate-binding protein, e.g., a lectin; C-type lectin receptors; glycoproteins; pattern recognition receptors (PRRs); peptidoglycan binding proteins; glycoprotein-binding molecules; amino acids; carbohydrates (including mono-, di-, tri- and poly-saccharides); lipids; steroids; hormones; lipid-binding molecules; cofactors; nucleosides; nucleotides; nucleic acids (e.g., DNA or RNA, analogues and derivatives of nucleic acids, or aptamers); peptidoglycan; lipopolysaccharide; cell surface receptors; and any combinations thereof.

In some embodiments the capture agent is an antibody. As used herein, the terms “antibody”, “antibodies” and “Ab” refer to intact antibody, or a binding fragment thereof that competes with the intact antibody for specific binding, and include polyclonal antibodies, monoclonal antibodies, humanized or chimeric antibodies, fully human antibodies, bispecific antibodies, single chain Fv antibody fragments, Fab fragments, Fab′ fragments, F(ab′)₂ fragments and F(ab)₂ fragments. Antibodies having specific binding affinity for a target analyte, such as histidine, can be produced through standard methods, such as described in the examples section. In some embodiments, binding fragments are produced by recombinant DNA techniques. In additional embodiments, binding fragments are produced by enzymatic or chemical cleavage of intact antibodies. Unless it is specifically noted, as used herein a “fragment thereof” in reference to an antibody refers to an immune specific fragment, i.e., a histamine-specific or binding fragment. In some embodiments, the anti-body is a commercially available antibody, such as an anti-histamine antibody. Without limitations, the antibodies can be monoclonal or polyclonal antibodies. In some embodiments, the capture agent is a chimeric antibody.

Polyclonal antibodies are heterogeneous populations of antibody molecules that are specific for a particular antigen, which are contained in the sera of the immunized animals. Polyclonal antibodies are produced using well-known methods. A chimeric antibody is a molecule in which different portions are derived from different animal species, such as those having a variable region derived from a murine monoclonal antibody and a human immunoglobulin constant region. Chimeric antibodies can be produced through standard techniques. Antibody fragments that have specific binding affinity for a component of the complex can be generated by known techniques. For example, such fragments include, but are not limited to, F(ab′)₂ fragments that can be produced by pepsin digestion of the antibody molecule, and Fab fragments that can be generated by reducing the disulfide bridges of F(ab′)₂ fragments. Alternatively, Fab expression libraries can be constructed. See, for example, Huse et al., 1989, Science, 246: 1275. Single chain Fv antibody fragments are formed by linking the heavy and light chain fragments of the Fv region via an amino acid bridge (e.g., 15 to 18 amino acids), resulting in a single chain polypeptide. Single chain Fv antibody fragments can be produced through standard techniques. See, for example, U.S. Pat. No. 4,946,778.

In some embodiments, the antibody or antigen-binding fragment thereof is murine. In some embodiments, the antibody or antigen-binding fragment thereof is from rabbit. In some embodiments, the antibody or antigen-binding fragment thereof is from a rat. In other embodiments, the antibody or antigen binding fragment thereof is human. In some embodiments the antibody or antigen-binding fragment thereof is recombinant, engineered, humanized and/or chimeric.

Monoclonal antibodies, which are homogeneous populations of antibodies to a particular epitope contained within an antigen, can be prepared using standard hybridoma technology. In particular, monoclonal antibodies can be obtained by any technique that provides for the production of antibody molecules by continuous cell lines in culture such as described by the human B-cell hybridoma technique (Kohler, G. et al., Nature, 1975, 256:495; Kosbor et al., Immunology Today, 1983, 4:72; Cole et al., Proc. Natl. Acad. Sci. USA, 1983, 80:2026), and the EBV-hybridoma technique (Cole et al., Monoclonal Antibodies and Cancer Therapy, Alan R. Liss, Inc., 1983, pp. 77-96). Such antibodies can be of any immunoglobulin class including IgG, IgM, IgE, IgA, IgD, and any subclass thereof. The hybridoma producing the monoclonal antibodies of the invention can be cultivated in vitro or in vivo.

In some embodiments antibodies can be commercial antibodies. Without limitation, some examples include: Ab 1 (GTX 12894) and Ab 2 (MAB5408) mouse monoclonal IgA antibody from Genetex and Millipore respectively, Ab 3 (MAB5408) an IgG mouse monoclonal antibody from Cloudclone; Ab 4 (PAA927Ge01), Ab 5 (H5080-06A), Ab 6 (GTX12840), Ab 7 (H7403) polyclonal rabbit IgG antibodies from Cloudclone, US biological, Genetex and Sigma respectively; Anti-mouse IgG-HRP (115-035-008) and anti-rabbit IgG (111-035-008) can be purchased from Jackson Immuno Research Laboratories while, anti-IgA HRP can be purchased from Invitrogen (62-6720).

In some embodiments the capture agent includes a detectable label. As used herein, a “detectable label” refers to a composition capable of producing a detectable signal indicative of the presence of a target, for example the conjugate in a capture zone 62. Detectable labels include any molecule or composition detectable by spectroscopic, photochemical, biochemical, immunochemical, electrical, optical or chemical means. Suitable labels include fluorescent molecules, radioisotopes, nucleotide chromophores, enzymes, substrates, chemiluminescent moieties, bioluminescent moieties, and the like. As such, a detectable label is any composition detectable by spectroscopic, photochemical, biochemical, immunochemical, electrical, optical or chemical means needed for the methods and devices described herein.

As used herein “detectable signal” can be a signal that is detected directly, such as by eye. For example, in some embodiments the detectable signal is seen as a color, an intensity or brightness, a color gradient between spots, or an intensity gradient between spots. In some embodiments the detectable signal refers to a processed signal which is quantified and can be give as a value, such as a numerical value. In some embodiments the detectable signals are combined, for example, where the quantified signal values are averaged or summed.

In some embodiments, the detectable label can be an echogenic substance (either liquid or gas), non-metallic isotope, an optical reporter, a boron neutron absorber, a paramagnetic metal ion, a ferromagnetic metal, a gamma-emitting radioisotope, a positron-emitting radioisotope, or an x-ray absorber.

Suitable optical reporters include, but are not limited to, fluorescent reporters and chemiluminescent groups. A wide variety of fluorescent reporter dyes are known in the art. Typically, the fluorophore is an aromatic or heteroaromatic compound and can be a pyrene, anthracene, naphthalene, acridine, stilbene, indole, benzindole, oxazole, thiazole, benzothiazole, cyanine, carbocyanine, salicylate, anthranilate, coumarin, fluorescein, rhodamine or other like compound.

Exemplary fluorophores include, but are not limited to, 1,5 IAEDANS; 1,8-ANS; 4-Methylumbelliferone; 5-carboxy-2,7-dichlorofluorescein; 5-Carboxyfluorescein (5-FAM); 5-Carboxynapthofluorescein (pH 10; 5-Carboxytetramethylrhodamine (5-TAMRA); 5-FAM (5-Carboxyfluorescein); 5-Hydroxy Tryptamine (HAT); 5-ROX (carboxy-X-rhodanine); 5-TAMRA (5-Carboxytetramethylrhodamine); 6-Carboxyrhodamine 6G; 6-CR 6G; 6-JOE; 7-Amino-4-methylcoumarin; 7-Aminoactinomycin D (7-AAD) 7-Hydroxy-4-methylcoumarin; 9-Amino-6-chloro-2-methoxyacridine; ABQ; Acid Fuchsin; ACMA (9-Amino-6-chloro-2-methoxyacridine); Acridine Orange; Acridine Red; Acridine Yellow; Acriflavin; Acrilavin Feulgen SITSA; Aequorin (Photoprotein); Alexa Fluor 350™; Alexa Fluor 430™; Alexa Fluor 488™; Alexa Fluor 532™; Alexa Flor 546™; Alexa Fluor 568™; Alexa Fluor 594™; Alexa Fluor 633™; Alexa Fluor 647™; Alexa Fluor 660™; Alexa Fluor 680™; Alizarin Complexon; Alizarin Red; Allophycocyanin (APC); AMC, AMCA-S; AMCA (Aminomethylcoumarin); AMCA-X; Aminoactinomycin D; Aminocoumarin; Anilin Blue; Anthrocyl stearate; APC-Cy7; APTS; Astrazon Brilliant Red 4G; Astrazon Orange R, Astrazon Red 6B; Astrazon Yellow 7 GLL; Atabrine; ATTO-TAG™ CBQCA; ATTO-TAG™ FQ; Auranine; Aurophosphine G; Aurophosphine; BAO 9 (Bisaminophenyloxadiazole); BCECF (high pH); BCECF (low pH); Berberine Sulphate; Beta Lactamase; BFP blue shifted GFP (Y66H); BG-647; Bimane; Bisbenzamide; Blancophor FFG; Blancophor SV; BOBO™-1; BOBO™-3; Bodipy 492/515; Bodipy 493/503; Bodipy 500/510; Bodipy 505/515; Bodipy 530/550; Bodipy 542/563; Bodipy 558/568; Bodipy 564/570; Bodipy 576/589; Bodipy 581/591; Bodipy 630/650-X; Bodipy 650/665-X, Bodipy 665/676; Bodipy FI; Bodipy FL ATP; Bodipy FI-Ceramide; Bodipy R6G SE; Bodipy TMR; Bodipy TMR-X conjugate; Bodipy TMR-X, SF; Bodipy TR; Bodipy TR ATP; Bodipy TR-X SE; BO-PRO™-1; BO-PRO™-3; Brilliant Sulphoflavin FF; Calcein; Calcein Blue; Calcium Crimson™; Calcium Green; Calcium Green-1 Ca²⁺ Dye; Calcium Green-2 Ca²⁺; Calcium (Green-5N Ca²⁺; Calcium Green-C18 Ca²⁺; Calcium Orange; Calcofluor White, Carboxy-X-rhodamine (5-ROX); Cascade Blue™; Cascade Yellow; Catecholamine; CFDA; CFP-Cyan Fluorescent Protein; Chlorophyll; Chromomycin A; Chromomycin A, CMFDA; Coelenterazine; Coelenterazine ep; Coelenterazine f; Coelenterazine fcp; Coelenterazine h; Coelenterazine hcp; Coelenterazine ip; Coelenterazine 0; Coumarin Phalloidin; CPM Methylcoumarin; CTC; Cy2™; Cy3.18; Cy3.5™; Cy3™; Cy5.1 8; Cy5.5™; Cy5™; Cy7™; Cyan GFP; cyclic AMP Fluorosensor (FiCRhR); d2; Dabcyl; Dansyl; Dansyl Amine; Dansyl Cadaverine; Dansyl Chloride; Dansyl DHPE; Dansyl fluoride; DAPI; Dapoxyl; Dapoxyl 2; Dapoxyl 3; DCFDA, DCFH (Dichlorodihydrofluorescein Diacetate); DDAO; DHR. (Dihydorhodanine 123); Di-4-ANEPPS; Di-8-ANEPPS (non-ratio); DiA (4-Di-16-ASP); DIDS; Dihydorhodamine 123 (DHR); DiO (DiOC18(3)); DiR; DiR (DiIC18(7)); Dopamine; DsRed; DTAF; DY-630-NHS; DY-635-NHS; EBFP; ECFP; EGFP; ELF 97; Eosin Erythrosin; Erythrosin ITC; Ethidium homodimer-1 (EthD-1); Euchlysin; Europium (III) chloride; Europium; EYFP; Fast Blue, FDA; Feulgen (Pararosaniline); FITC, FL-645; Flazo Orange; Fluo-3; Fluo-4; Fluorescein Diacetate; Fluoro-Emerald; Fluoro-Gold (Hydroxystilbamidine); Fluor-Ruby; FluorX; FM 1-43™; FM 4-46; Fura Red™ (high pH); Fura-2, high calcium; Fura-2, low calcium, Genacryl Brilliant Red B; Genacryl Brilliant Yellow 10GF; Genacryl Pink 3G; Genacryl Yellow 5GF; GFP (S65T); GFP red shifted (rsGFP); GFP wild type, non-UV excitation (wtGFP); GFP wild type, UV excitation (wtGFP); GFPuv; Gloxalic Acid; Granular Blue; Haematoporphyrin; Hoechst 33258; Hoechst 33342; Hoechst 34580, HPTS; Hydroxycoumarin; Hydroxystilbamidine (FluoroGold); Hydroxytryptamine; Indodicarbocyanine (DiD); Indotricarbocyanine (DiR); Intrawhite Cf; JC-1; JO-JO-1; JO-PRO-1; LaserPro; Laurodan; LDS 751; Leucophor PAF; Leucophor SF; Leucophor WS; Lissamine Rhodamine; Lissanine Rhodanine B; LOLO-1; LO-PRO-1; Lucifer Yellow; Mag Green, Magdala Red (Phloxin B); Magnesium Green; Magnesium Orange; Malachite Green; Marina Blue; Maxilon Brilliant Flavin 10 GFF; Maxilon Brilliant Flavin 8 GFF; Merocyanin; Methoxycoumarin; Mitotracker Green FM; Mitotracker Orange; Mitotracker Red; Mitramycin; Monobromobimane; Monobromobimane (mBBr-GSH); Monochlorobimane; MPS (Methyl Green Pyronine Stilbene); NBD; NBD Amine; Nile Red; Nitrobenzoxadidole; Noradrenaline; Nuclear Fast Red, Nuclear Yellow; Nylosan Brilliant lavin E8G; Oregon Green™, Oregon Green 488-X; Oregon Green™ 488; Oregon Green™ 500; Oregon Green™ 514; Pacific Blue; Pararosaniline (Feulgen); PE-Cy5, PE-Cy7; PerCP; PerCP-Cy5.5; PE-TexasRed (Red 613); Phloxin B (Magdala Red); Phorwite AR; Phorwite BK L; Phorwite Rev, Phorwite RPA, Phosphine 3R; PhotoResist; Phycoerythrin B [PE]; Phycoerythrin R [PE]; PKH26; PKH67, PMIA, Pontochrome Blue Black; POPO-1; POPO-3; PO-PRO-1; PO-PRO-3; Primuline; Procion Yellow; Propidium Iodid (PI); PyMPO; Pyrene; Pyronine; Pyronine B; Pyrozal Brilliant Flavin 7GF; QSY 7; Quinacrine Mustard; Resorufin; RH 114; Rhod-2; Rhodamine; Rhodamine 110; Rhodamine 123; Rhodanine 5 GLD; Rhodamine 6G; Rhodanine B 540, Rhodamine B 200; Rhodanine B extra; Rhodamine BB; Rhodamine BG; Rhodamine Green; Rhodamine Phallicidine; Rhodamine Phalloidine; Rhodamine Red; Rhodamine WT; Rose Bengal; R-phycoerythrin (PE); red shifted GFP (rsGFP, S65T); S65A; S65C; S65L; S65T; Sapphire GFP; Serotonin; Sevron Brilliant Red 2B; Sevron Brilliant Red 4G; Sevron Brilliant Red B; Sevron Orange; Sevron Yellow L; sgBFP™; sgBFP™ (super glow BFP); sgGFP™; sgGFP™ (super glow GFP); SITS; SITS (Primuline); SITS (Stilbene Isothiosulphonic Acid); SPQ (6-methoxy-N-(3-sulfopropyl)-quinolinium); Stilbene; Sulphorhodamine B can C; Sulphorbodamine (G Extra; Tetracycline; Tetramethylrhodamine; Texas Red™; Texas Red-X™ conjugate; Thiadicarbocyanine (DiSC3); Thiazine Red R; Thiazole Orange; Thioflavin 5; Thioflavin S; Thioflavin TCN; Thiolyte; Thiozole Orange; Tinopol CBS (Calcofluor White); TMR; TO-PRO-1, TO-PR-O-3; TO-PRO-5; TOTO-1; TOTO-3; TriColor (PE-Cy5); TRITC (TetramethylRodaminelsoThioCyanate); True Blue; TruRed; Ultralite; Uranine B; Uvitex SFC; wt GFP; WW 781; XL665; X-Rhodamine; XRITC; Xylene Orange; Y66F; Y66H; Y66W; Yellow GFP, YFP, YO-PRO-1; YO-PRO-3; YOYO-L, and YOYO-3 Many suitable forms of these fluorescent compounds are available and can be used.

Other exemplary detectable labels include luminescent and bioluminescent markers (e.g., biotin, luciferase (e.g., bacterial, firefly, click beetle and the like), luciferin, and aequorin), radiolabels (e.g., 3H, 125I, 35S, 14C, or 32P), enzymes (e.g., galactosidases, glucuronidases, phosphatases (e.g., alkaline phosphatase), peroxidases (e.g., horseradish peroxidase), and cholinesterases), and calorimetric labels such as colloidal gold or colored glass or plastic (e.g., polystyrene, polypropylene, and latex) beads. Patents teaching the use of such labels include U.S. Pat. Nos. 3,817,837, 3,850,752, 3,939,350, 3,996,345, 4,277,437, 4,275,149, and 4,366,241, each of which is incorporated herein by reference.

Suitable non-metallic isotopes include, but are not limited to, ¹¹C, ¹⁴C, ¹³N, ¹⁸F, ¹²³I, ¹²⁴I, and ¹²⁵I. Suitable radioisotopes include, but are not limited to, ⁹⁹mTc, ⁹⁵Tc, ¹¹¹In, ⁶²Cu, Ga, ⁶⁸Ga, and ¹⁵³Gd. Suitable paramagnetic metal ions include, but are not limited to, Gd(II), Dy(III), Fe(III), and Mn(II). Suitable X-ray absorbers include, but are not limited to, Re, Sm, Ho, Lu, Pm, Y, Bi, Pd, Gd, La, Au, Au, Yb, Dy, Cu, Rh, Ag, and Ir.

Suitable non-metallic isotopes include, but are not limited to, ¹¹C, ¹⁴C, ¹³N, ¹⁸F, ¹²³I, ¹²⁴I, and ¹²⁵I. Suitable radioisotopes include, but are not limited to, ⁹⁹mTc, ⁹⁵Tc, ¹¹¹In, ⁶²Cu, ⁶⁴Cu, Ga, ⁶⁸Ga, and ¹⁵³Gd. Suitable paramagnetic metal ions include, but are not limited to, Gd(III), Dy(III), Fe(III), and Mn(II). Suitable X-ray absorbers include, but are not limited to, Re, Sm, Ho, Lu, Pm, Y, Bi, Pd, Gd, La, Au, Au, Yb, Dy, Cu, Rh, Ag, and Ir. In some embodiments, the radionuclide is bound to a chelating agent or chelating agent-linker attached to the microbe-targeting molecule. Suitable radionuclides for direct conjugation include, without limitation, ¹⁸F, ¹²⁴I, ¹²⁵I, ¹³¹I, and mixtures thereof. Suitable radionuclides for use with a chelating agent include, without limitation, ⁴⁷Sc, ⁶⁴Cu, ⁶⁷Cu, ⁸⁹Sr, ⁸⁶Y ⁸⁷Y, ⁹⁰Y, ¹⁰⁵Rh, ¹¹¹Ag, ¹¹¹In, ¹¹⁷mSn, ¹⁴⁹Pm, ¹⁵³Sm, ¹⁶⁶Ho, ¹⁷⁷Lu, ¹⁸⁶Re, ¹⁸⁸Re, ²¹¹At, ²¹²Bi, and mixtures thereof. Suitable chelating agents include, but are not limited to, DOTA, BAD, TETA, DTPA, EDTA, NTA, HDTA, their phosphonate analogs, and mixtures thereof. One of skill in the art will be familiar with methods for attaching radionuclides, chelating agents, and chelating agent-linkers to molecules such as the microbe-targeting molecules and carrier scaffolds disclosed herein.

Means of detecting such labels are well known to those of skill in the art. Thus, for example, radiolabels can be detected using photographic film or scintillation counters, fluorescent markers can be detected using a photo-detector to detect emitted light. Enzymatic labels are typically detected by providing the enzyme with an enzyme substrate and detecting the reaction product produced by the action of the enzyme on the enzyme substrate, and calorimetric labels can be detected by visualizing the colored label.

In some embodiments, the detectable label is a fluorophore or a quantum dot. Without wishing to be bound by a theory, using a fluorescent reagent can reduce signal-to-noise in the imaging/readout, thus maintaining sensitivity.

Any method known in the art for detecting the particular label can be used for detection. Exemplary methods include, but are not limited to, spectrometry, fluorometry, microscopy imaging, immunoassay, and the like.

In some embodiments the detectable label is a nanoparticle. For example, a gold nanoparticle. In some embodiments the capture agent is attached to the nanoparticle and can be detected on the LFA at the capture zones 62. As the amount of nanoparticle accumulates, the intensity or contrast of the dot increases and can be distinguished spectroscopically or by eye. For example, some gold nanoparticles form a red spot with increasing intensity proportional to the accumulated concentration. The color is dependent on the size of the nanoparticle and some embodiments include particles with different corresponding colors such as yellow, green, purple. In some embodiments, images can be take of the detection zone 60 and the dots analyzed, for example, by using an appropriate computer and monitor using an image processing software. For example, in some embodiments the imaging software can be ImageJ which is available at www.imagej.nih.gov/ij, accessed Jul. 24, 2019.

As used herein “nanoparticle” is not limited to a particular shape and size and can include spherical, rod like, faceted, plates or other shapes, and can be monodisperse or polydisperse. The sizes can vary such as between about 1 nm and about 1000 nm, such as between about 2 and about 500 nm, or 10 and about 100 nm. In some embodiments the nanoparticles are monodisperse. In some embodiments the nanoparticles have a narrow particle size distribution such as having a polydispersity index below about 0.5, such as below about 0.4, below about 0.3 or below about 0.2. Methods of conjugating nanoparticles to other molecules are well known in the art.

The conjugate and the analyte competitively bind with the capture agent. Generally, the analyte has a higher binding affinity than the conjugate for binding with the capture agent, and therefore the analyte can displace a capture agent that is bound to the conjugate. As used herein, the term “binding affinity” refers to the strength of the sum total of noncovalent interactions between a single binding site of composition or molecule such as the capture agent (e.g., an antibody) and its binding partner such as the analyte or the competitive molecule (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (Kd). Lower Kd means higher binding affinity. Accordingly, in some embodiments, the analyte has a lower dissociation constant than the conjugate for binding with the capture agent. For example, the Kd of the competitive molecule binding to the capture agent can be at least 1.1×, 1.2×, 1.25×, 1.5×, 2×, 2.5×, 5×, 10×, 25×, 50×, 100× or higher than the Kd of the analyte binding to the same capture agent.

A variety of methods of measuring binding affinity are known in the art and can be used to measure the binding affinity and dissociation rate of a capture agent for use in the devices and methods described herein. For example, the binding affinity can be measured by competitive ELISAs, RIAs, BIACORE™, or KINEXA™ technology. The dissociation rate also can be measured by BIACORE™ or KINEXA™ technology. For example, the binding affinity and dissociation rate of a capture agent, such as an antibody can be measured by surface plasmon resonance using, e.g., a BIACORE™ system (e.g., a BIACORE™-2000 or a BIACORE™-3000 (BIAcore, Inc., Piscataway, N.J.) at 25° C. with immobilized antigen, e.g., analyte or competitive molecule chips at about 10 response units (RU).

As used herein, the term “small molecules” refers to natural or synthetic molecules including, but not limited to, amino acids, peptides, peptidomimetics, polynucleotides, aptamers, nucleotide analogs, organic or inorganic compounds (i.e., including heterorganic and organometallic compounds), saccharides (e.g., mono, di, tri and polysaccharides), steroids, hormones, pharmaceutically derived drugs (e.g., synthetic or naturally occurring), lipids, derivatives of these (e.g., esters and salts of these), fragments of these, and conjugates of these. In some embodiments the small molecules have a molecular weight less than about 10,000 Da, less than about 5,000 Da, less than about 1,000 Da, less than about 500 Da. In some embodiments the small molecule has a molecular weight of less than about 1000 Da.

In some embodiments, the small molecule is an amino acid or nucleoside that has been modified. For example, without limitation, amino acid and nucleoside modifications can include acetylation, glycosylation, amidation, hydroxylation, methylation, ubiquitylation, pyrrolidon carboxylic acid, sulfation, racemization, isomerization, phosphorylation, cyclization, sumoylation, formation of disulfide bridges, deamidation, deamination, eliminylation, oxidation, reduction, pegylation, and combinations of these.

In some embodiments the small molecule comprises an imidazole group. In some embodiments, the small molecule is histamine or histidine. In some other embodiments the small molecule is a substituted aromatic compound such as dinitrophenol (e.g., 2, 4-dinitrophenol). In some embodiments the small molecule is a steroid such as cortisol.

As described herein, the conjugate is attached, covalently or non-covalently, to the surface of the LFA providing an immobilized conjugate. As used herein “immobilized” means the conjugate will not flow with solutions or fluids in the LFA although the conjugate can flex and extend from its point of attachment to the LFA surface. Generally, the conjugate comprises one or more analyte-related molecules conjugated to the lateral flow matrix via a linker.

In some embodiments a control zone 64 or a control zone 64′ includes an analyte-related (e.g., a target analyte molecule) molecule immobilized on the LFA. In some embodiments the analyte-related molecule in the capture zone 62, and the analyte-related molecule immobilized in the control zone 64 or 64′ are different molecules. In some other embodiments, the analyte-related molecule and the analyte are same.

As used herein the analyte-related molecule is a molecule that is the same or similar to the small molecule analyte except that it is attached to the conjugate. In some embodiments, the analyte-related molecule has a molecular weight that is not more than or less than 20%, the of the analyte. In some embodiments the analyte-related molecule has one or more atomic isomers enhanced as compared to the analyte molecule, such as deuterium replacing any hydrogen, or ¹³C replacing any carbon (natural distribution). In some embodiments, the analyte-related molecule includes a substituted atom from the same family as compared to the analyte, such as sulphur replacing oxygen, or arsenic replacing nitrogen. Without being bound to any specific theory the analyte-related molecule presents a similar topography and functional groups to the capture agent as the analyte molecule.

As used in this disclosure, the term “linker” means a moiety that can directly or indirectly connect two parts of a compound, molecule or composition. Linkers typically comprise a direct bond or an atom such as oxygen or sulfur, a unit such as NR¹, C(O), C(O)O, OC(O)O, C(O)NH, NHC(O)O, NH, SS, SO, SO₂, SO₃, and SO₂NH, or a chain of atoms, such as substituted or unsubstituted alkyl, substituted or unsubstituted alkenyl, substituted or unsubstituted alkynyl, arylalkyl, arylalkenyl, arylalkynyl, heteroarylalkyl, heteroarylalkenyl, heteroarylalkynyl, heterocyclylalkyl, heterocyclylalkenyl, heterocyclylalkynyl, aryl, heteroaryl, heterocyclyl, cycloalkyl, cycloalkenyl, alkylarylalkyl, alkylarylalkenyl, alkylarylalkynyl, alkenylarylalkyl, alkenylarylalkenyl, alkenylarylalkynyl, alkynylarylalkyl, alkynylarylalkenyl, alkynylarylalkynyl, alkylheteroarylalkyl, alkylheteroarylalkenyl, alkylheteroarylalkynyl, alkenylheteroarylalkyl, alkenylheteroarylalkenyl, alkenylheteroarylalkynyl, alkynylheteroarylalkyl, alkynylheteroarylalkenyl, alkynylheteroarylalkynyl, alkylheterocyclylalkyl, alkylheterocyclylalkenyl, alkylhererocyclylalkynyl, alkenylheterocyclylalkyl, alkenylheterocyclylalkenyl, alkenylheterocyclylalkynyl, alkynylheterocyclylalkyl, alkynylheterocyclylalkenyl, alkynylheterocyclylalkynyl, alkylaryl, alkenylaryl, alkynylaryl, alkylheteroaryl, alkenylheteroaryl, alkynylhereroaryl, where one or more methylenes can be interrupted or terminated by O, S, S(O), SO₂, NH, C(O)N(R¹)₂, C(O), cleavable linking group, substituted or unsubstituted aryl, substituted or unsubstituted heteroaryl, substituted or unsubstituted heterocyclic; where R¹ is hydrogen, acyl, aliphatic or substituted aliphatic. In some embodiments, the linking effectuated by the linker can be by a non-covalent association (e.g., by non-covalent interactins) of the two parts of a molecule being conjugated together. Some exemplary non-covalent on ionic interactions, van der Waals interactions, dipole-dipole interactions, hydrogen bonds, electrostatic interactions, and/or shape recognition interactions.

The linkers can be of any shape. For example, the linker can be linear, folded, branched. In some embodiments, the linkers can be linear. In some embodiments, the linker can be branched. For branched linkers, the linker can link together at least one (e.g., one, two, three, four, five, six, seven, eight, nine, ten or more) competitive molecules or analyte molecules.

Linkers can be configured according to a specific need, e.g., based on at least one of the following characteristics. By way of example only, in some embodiments, linkers can be configured to have a sufficient length and flexibility such that it can allow for an analyte-related molecule to orient accordingly with respect to a receptor site of a large molecule such as an antigen-binding site of an antibody. In some embodiments the linker can include flexible structure units such polyethylene, poly ethylene glycol or poly propylene glycol groups. In some embodiments the linking groups have a medium to high solubility in aqueous solutions. Without being bound by any specific theory, this solubility, or affinity for water, allows the linker to extend into an aqueous solution rather than self-associate. In some other embodiments, a linker can be selected to be compatible with non-aqueous solutions, such as hydrocarbons and fluorocarbons, e.g., thereby extending into these solutions rather than self-associating. In some embodiments the linker is non-toxic. In some embodiments the linker does not react or bind to a sensing antibody or components of a patient sample such as blood, plasma, semen, mucus and other biological fluids. In some embodiments the linker can be any linking group as described in U.S. Pat. No. 5,112,738 which is hereby incorporated by reference. For example, the linker can be linear or branched alkenes comprising from 1 to as many as 40 (e.g., as many as 30 or 20), or 2, 6, 8, 10 to as many as 20, (i.e., methylene, ethylene, n-propylene, iso-propylene, n-butylene, and so forth). In addition, such alkylenes can contain other substituent groups such as cyano, amino (including substituted amino), acylamino, halogen, thiol, hydroxyl, carbonyl groups, carboxyl (including substituted carboxyls such as esters, amides, and substituted amides). The linker can also contain or consist of substituted or unsubstituted aryl, aralkyl, or heteroaryl groups (e.g., phenylene, phenethylene, and so forth). Additionally, such linkers can contain one or more heteroatoms selected from nitrogen, sulfur and oxygen in the form of ether, ester, amido, amino, thio ether, amidino, sulfone, or sulfoxide. Also, such linkers can include unsaturated groupings such as olefinic or acetylenic bonds, imino, or oximino groups. In some embodiments the linker will be a chain, such as aliphatic comprising between 6 and about 60 atoms excluding hydrogen, between 6 and 50, between 6 and 40, between 6 and 30, between 6 and 20, between 6 and 10, of which between 0 and 60 atm % (e.g., 0 and 50 atm %, 0 and 40 atm %, 10 and 40 atm %) are heteroatoms selected from nitrogen, oxygen, and sulfur.

In some embodiments the linker comprises a polyethylene glycol with between about 2 and 45 repeat units (e.g., between about 2 and 30 repeat units, between about 2 and 20 repeat units, between about 4 and 10 repeat units). As used herein Poly(ethylene glycol) (PEG), polyethylene glycol, poly(oxyethylene) or poly(ethylene oxide) (PEO), are used interchangeably. Where PEG(x) is used, x is the approximate molecular weight of the linker group. In some other embodiments the linking group comprises polypropylene groups with between 2 and 45 repeat units (e.g., between about 2 and 30 repeat units, between about 2 and 20 repeat units, between about 4 and 10 repeat units). Optionally, the linker has a length between 5 and 200 angstroms. For example, the linker length is greater than about 5 and less than about 200 Å (e.g., greater than 5 Å and less than about 180 Å, greater than about 7 Å and less than about 157.5 Å, between about 7 Å and about 100 Å).

In some other embodiments, without limitations, the linker comprises a polyamide, polyimide, polytetrafluoroethylene, polyurethane, polyesters, polyols, polysaccharides, peptides, polyacrylonitrile, RNA, DNA or a fragment comprising between 2 and 30 repeat units of these polymers (e.g., a dimer, trimer or oligomer). In some embodiments the linker is not a protein or peptide.

In some embodiments the linker comprises a polymer chain (branched or linear). In some embodiments, chemical linkers can comprise a direct bond or an atom such as oxygen or sulfur, a unit such as NH, C(O), C(O)NH, SO, SO₂, SO₂NH, or a chain of atoms, such as substituted or unsubstituted C₁-C₆ alkyl, substituted or unsubstituted C₂-C₆ alkenyl, substituted or unsubstituted C₂-C₆ alkynyl, substituted or unsubstituted C₆-C₁₂ aryl, substituted or unsubstituted C₅-C₁₂ heteroaryl, substituted or unsubstituted C₅-C₁₂ heterocyclic, substituted or unsubstituted C₃-C₁₂ cycloalkyl, where one or more methylenes can be interrupted or terminated by O, S, S(O), SO₂, NH, or C(O).

In some embodiments, the linker can comprise a one or more terminal or internal group(s) for attachment to the substrate. For example, the linker can comprise one or more of O, S, S(O), SO₂, NH, or C(O) groups for attachment to the substrate. In some embodiments, the linker comprises at least one amino group that can non-covalently or covalently couple with functional groups on the surface of the substrate. For example, the primary amines at the N-terminus or in close proximity to the N-terminus of the linker can be used to couple with functional groups on the substrate surface. In some embodiments the one or more O, S, S(O), SO₂, NH, or C(O) are part of a group forming a link to the analyte-related or the branching domain. For example, an ester bond (—NHC(O)—) formed by the reaction of an amino group on a PEG based linker with a carboxylic acid from a competitive molecule, an analyte or a branching domain.

A “binding pair”, “coupling molecule pair” and “coupling pair” are used interchangeably and without limitation herein to refer to the first and second molecules or functional groups that specifically bind to each other. For example, the binding can be through one or more of a covalent bond, a hydrogen bond, an ionic bond, and a dative bond. In some embodiments one member of the binding pair is conjugated with a solid substrate while the second member is conjugated with the linker. A binding pair can be used for linking the linker to the substrate, and/or for linking the linker to the analyte-related molecule.

Exemplary coupling molecule pairs also include, without limitations, any haptenic or antigenic compound in combination with a corresponding antibody or binding portion or fragment thereof (e.g., digoxigenin and anti-digoxigenin; mouse immunoglobulin and goat antimouse immunoglobulin) and nonimmunological binding pairs (e.g., biotin-avidin, biotin-streptavidin), hormone (e.g., thyroxine and cortisol-hormone binding protein), receptor-receptor agonist, receptor-receptor antagonist (e.g., acetylcholine receptor-acetylcholine or an analog thereof), IgG-protein A, lectin-carbohydrate, enzyme-enzyme cofactor, enzyme-enzyme inhibitor, and complementary oligonucleotide pairs capable of forming nucleic acid duplexes). The coupling molecule pair can also include a first molecule that is negatively charged and a second molecule that is positively charged.

One example of using coupling pair conjugation is the biotin-avidin or biotin-streptavidin conjugation. In this approach, one of the members of the coupling pair (e.g., a portion of the engineered microbe-targeting molecule such as substrate-binding domain, or a substrate) is biotinylated and the other (e.g., a substrate or the engineered microbe-targeting molecule) is conjugated with avidin or streptavidin. Many commercial kits are also available for biotinylating molecules, such as proteins. For example, an aminooxy-biotin (AOB) can be used to covalently attach biotin to a molecule with an aldehyde or ketone group. In one embodiment, AOB is attached to the substrate-binding domain (e.g., comprising AKT oligopeptide) of the engineered microbe-targeting molecule.

One non-limiting example of using conjugation with a coupling molecule pair is the biotin-sandwich method. See, e.g., Davis et al., 103 PNAS 8155 (2006). The two molecules to be conjugated together are biotinylated and then conjugated together using tetravalent streptavidin. In addition, a peptide can be coupled to the 15-amino acid sequence of an acceptor peptide for biotinylation (referred to as AP; Chen et al., 2 Nat. Methods 99 (2005)). The acceptor peptide sequence allows site-specific biotinylation by the E. coli enzyme biotin ligase (BirA; Id.). An engineered microbe surface-binding domain can be similarly biotinylated for conjugation with a solid substrate. Many commercial kits are also available for biotinylating proteins. Another example for conjugation to a solid surface would be to use PLP-mediated bioconjugation. See, e.g., Witus et al., 132 JACS 16812 (2010).

Still another example of using coupling pair conjugation is double-stranded nucleic acid conjugation. In this approach, one of the members of the coupling pair (e.g., a portion of the engineered microbe-targeting molecule such as substrate-binding domain, or a substrate) can be conjugated with a first strand of the double-stranded nucleic acid and the other (e.g., a substrate) is conjugated with the second strand of the double-stranded nucleic acid. Nucleic acids can include, without limitation, defined sequence segments and sequences comprising nucleotides, ribonucleotides, deoxyribonucleotides, nucleotide analogs, modified nucleotides and nucleotides comprising backbone modifications, branchpoints and nonnucleotide residues, groups or bridges.

Other examples for forming a coupling pair include click chemistry. As used herein “click chemistry” refers to a class of small molecule reactions which can be used for the linking of a binding pair and is not a single specific reaction but rather describes the method of generating products by mimicking nature which produces substance by joining of small modular units. Although useful for biochemical reactions, click chemistry is not limited to biological conditions. Click reactions are efficient and easy to used, occurring in one pot without any special precautions against water and air, do not produce offensive (e.g., not toxic) byproducts, and, because they are characterized by a high thermodynamic driving force that drives the reaction quickly to a single reaction product, require minimal or no final isolation and purification. Examples of click chemistry includes the copper-catalyzed reaction of an azide with an alkyne to form a 5-membered heteroatom ring (e.g., a Cu(I)-catalyzed azide-alkyne cycloaddition), the thiol-Michael Addition reaction such as reaction of a thiol group with a maleimide group, strain-promoted azide-alkyne cycloaddition, strain-promoted alkyne-nitrone cycloaddition, reactions of strained alkenes, alkene and azide [3+2]cycloaddition, alkene and tetrazine inverse-demand Diels-Alder, and alkene and tetrazole photoclick reaction. In some embodiments, a coupling pair is formed using the reaction of a thiol group with a malamide group, forming a thiol-malamide link.

In other embodiments condensation reactions such as amide bond formation between and amine and carboxylic acids can be used to link the linker to the substrate or an analyte related molecule. In still other embodiments the coupling pair can include adsorption such as adsorption of a thiol to a gold surface. Embodiments can also include the reaction of alkyl halide, aldehyde, amino, bromo or iodoacetyl, carboxyl, hydroxyl, epoxy, ester, silane, thiol, and the like, wherein these groups can be one part of the binding pair. Other embodiments include ionic-boding wherein a positive and negative pair combine.

In some embodiments the linker comprises at least one lysine. In some embodiments, at least one analyte related molecule or analyte is linked to the alpha-amino group of the at least one lysine. In some additional embodiments, at least one analyte-related molecule or analyte is linked to the alpha-amino group of the at least one lysine and at least one analyte-related molecule or analyte is linked to the epsilon-amino group of the at least one lysine.

In some embodiments, the linker comprises a first lysine linked to a second lysine, and wherein the carboxyl group of the first lysine is linked to the epsilon-amino group of second lysine. In some other embodiments, the linker comprises a first lysine, a second lysine and a third lysine, and wherein the carboxyl group of the first lysine is linked to the epsilon-amino group of the second lysine, and the carboxyl group of the third lysine is linked to the alpha-amino group of the first or second lysine. In some embodiments, an available amino group of a lysine in the linker comprises an analyte-related molecule or analyte. For example, an analyte-related molecule or analyte can be linked to any available amino group in the linker comprising lysine(s).

In some embodiments, the linker comprises n lysines, wherein n is an integer greater than three (e.g., between about 3 and 100), wherein the carboxyl group of a first lysine (n=1 lysine) is linked to the epsilon-amino group of a second lysine (n=2 lysine), the carboxyl group of a third lysine (n=3 lysine) is linked to the alpha-amino group of the first or second lysine, the carboxyl group of the fourth lysine is linked to the alpha-amino group of the first, second, or third lysine, and the carboxylic group of the n^{th} lysine is linked to the alpha-amino group any one of the lysines up to the (n−1)^{th} lysine. Optionally, an analyte-related molecule or analyte can be linked to any available amino group in the linker comprising lysine(s).

In some embodiments, the linker comprises a polyethylene glycol and one or more lysine residues.

In some embodiments, the linker is a bond.

In some embodiments, the linker comprises the following structure:

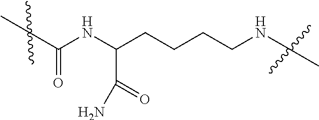 [SMILES: *NCCCCC(NC(*)=O)C(N)=O]

In some embodiments, the conjugate comprising the analyte-related molecule or the analyte comprises the following structure:

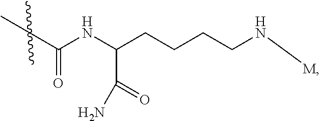

wherein M is an analyte related molecule or an analyte.

In some embodiments, the conjugate comprising the analyte-related comprises the compound having Structure 1:

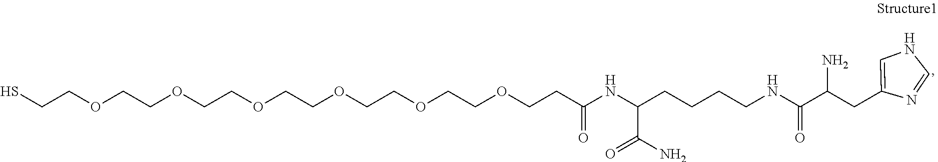 [SMILES: NC(=O)C(CCCCNC(=O)C(N)Cc1c[nH]cn1)NC(=O)CCOCCOCCOCCOCCOCCOCCS]

where the conjugate linked to the substrate via the thiol group.

In some embodiments, the linker comprises a structure selected from the following:

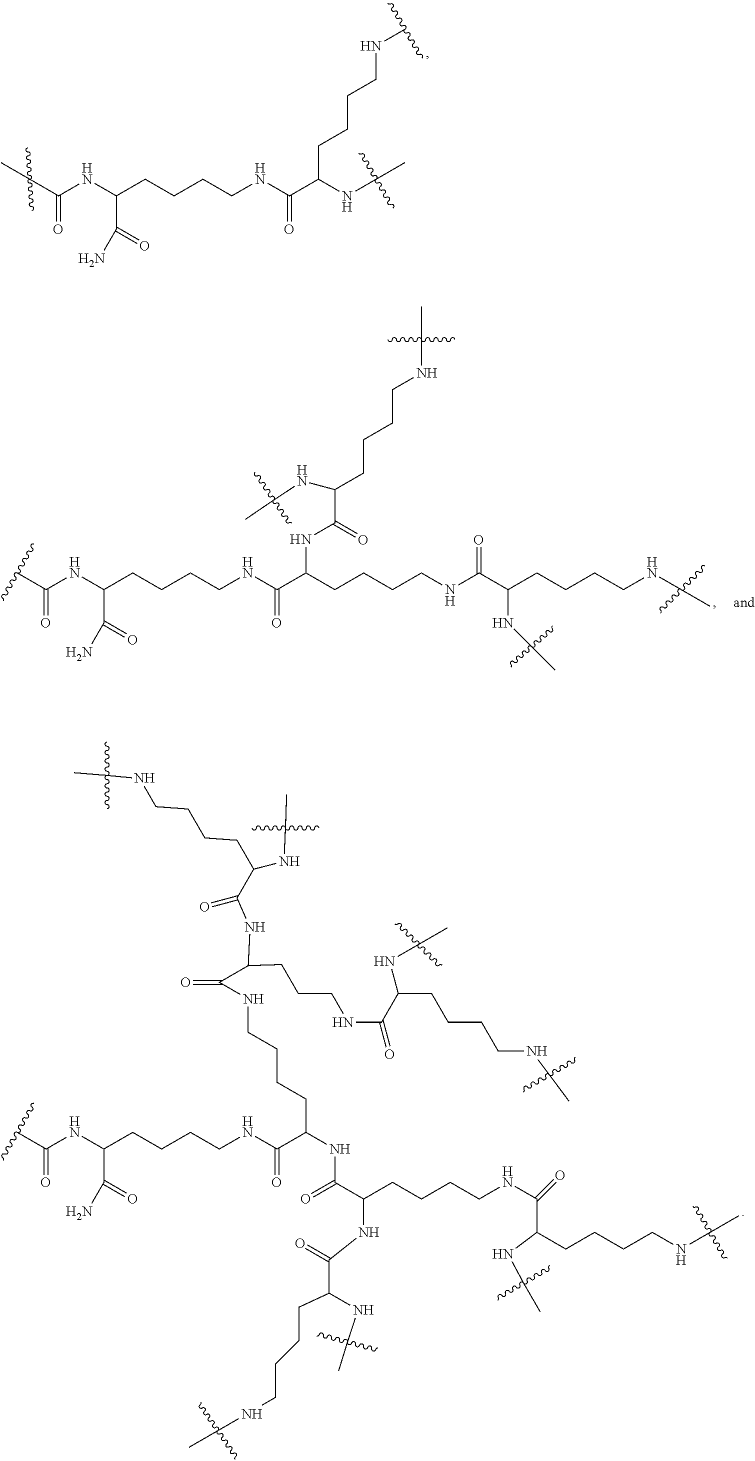 [SMILES: *NCCCCC(N*)C(=O)NCCCCC(NC(*)=O)C(N)=O ; *NCCCCC(N*)C(=O)NCCCCC(NC(=O)C(CCCCN*)N*)C(=O)NCCCCC(NC(*)=O)C(N)=O ; *NCCCCC(N*)C(=O)NCCCCC(NC(=O)C(CCCCN*)N*)C(=O)NC(CCCCNC(=O)C(CCCNC(=O)C(CCCCN*)N*)NC(=O)C(CCCCN*)N*)C(=O)NCCCCC(NC(*)=O)C(N)=O]

In some embodiments, the conjugate comprising the analyte-related molecule or the analyte comprises a structure selected from the following:

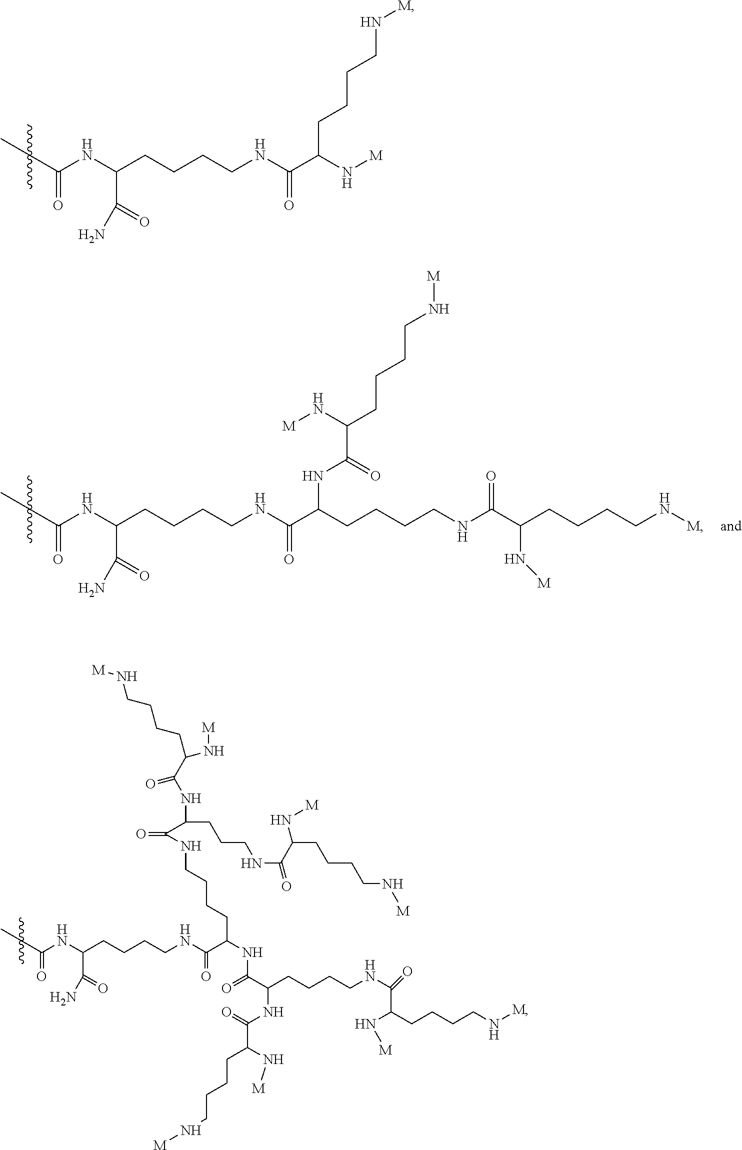

wherein each M is an analyte related molecule or an analyte. It is noted that if M is an analyte-related molecule then all M are independently selected analyte-related molecules.

In some embodiments, the conjugate comprising the analyte-related molecule or the analyte comprises the following structure:

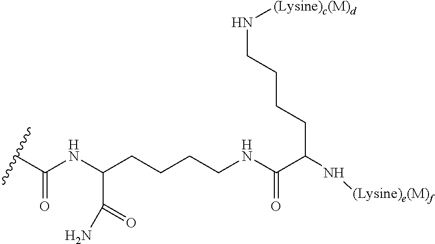

wherein, d+f≥2 (e.g., between about 2 and 100), d≥c, and e≥f, wherein c, d, e and f are integers and each M is an analyte-related molecule or analyte.

In some embodiments, the conjugate comprising the analyte-related molecule comprises the following structure:

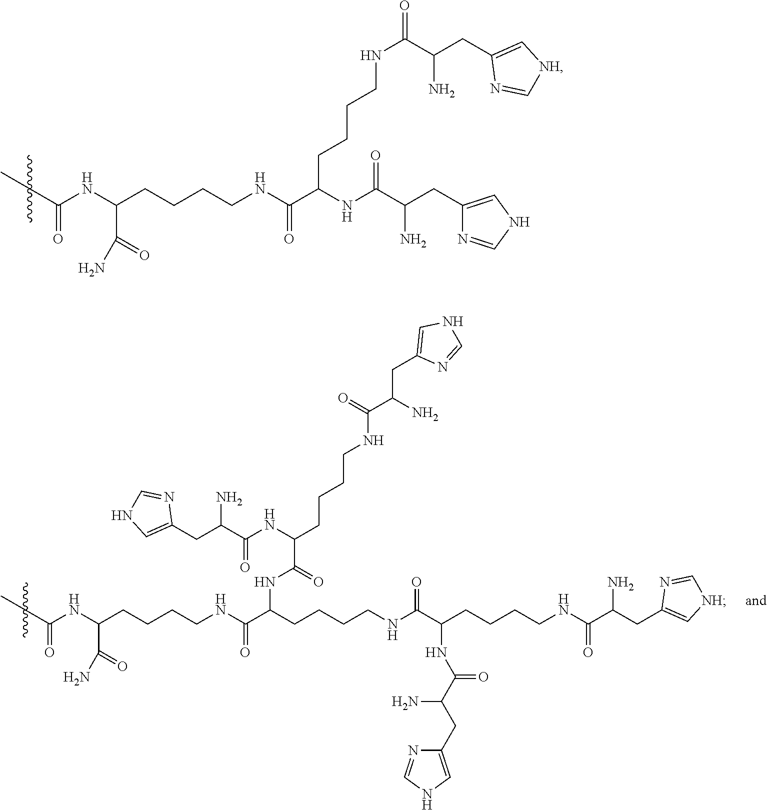 [SMILES: *C(=O)NC(CCCCNC(=O)C(CCCCNC(=O)C(N)Cc1c[nH]cn1)NC(=O)C(N)Cc1c[nH]cn1)C(N)=O ; *C(=O)NC(CCCCNC(=O)C(CCCCNC(=O)C(CCCCNC(=O)C(N)Cc1c[nH]cn1)NC(=O)C(N)Cc1c[nH]cn1)NC(=O)C(CCCCNC(=O)C(N)Cc1c[nH]cn1)NC(=O)C(N)Cc1c[nH]cn1)C(N)=O]

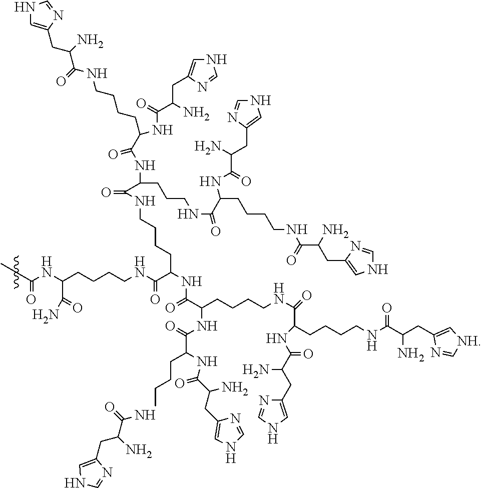 [SMILES: *C(=O)NC(CCCCNC(=O)C(CCCCNC(=O)C(CCCNC(=O)C(CCCCNC(=O)C(N)Cc1c[nH]cn1)NC(=O)C(N)Cc1c[nH]cn1)NC(=O)C(CCCCNC(=O)C(N)Cc1c[nH]cn1)NC(=O)C(N)Cc1c[nH]cn1)NC(=O)C(CCCCNC(=O)C(CCCCNC(=O)C(N)Cc1c[nH]cn1)NC(=O)C(N)Cc1c[nH]cn1)NC(=O)C(CCCCNC(=O)C(N)Cc1c[nH]cn1)NC(=O)C(N)Cc1c[nH]cn1)C(N)=O]

In some embodiments, the linker can comprise at least one, at least two, at last‘C’ three or more oligopeptides. The length of the oligonucleotide can vary from about 2 amino acid residues to about 10 amino acid residues, or about 2 amino acid residues to about 5 amino acid residues. Determination of an appropriate amino acid sequence of the oligonucleotide for binding with different substrates is well within one of skill in the art. For example, an oligopeptide comprising an amino acid sequence of Alanine-Lysine-Threonine (AKT), which provides a single biotinylation site for subsequent binding to streptavidin-coated substrate.

It is noted that a conjugate described herein can be directly or indirectly linked to a carrier scaffold for immobilizing on the lateral flow matrix. As used herein, a “carrier scaffold”: means a compound, molecule or composition that can directly or indirectly immobilize the conjugate on to the lateral flow matrix. Some exemplary carrier scaffolds include, but are not limited to, peptides, proteins, nucleic acids, and the like.

In some embodiments, the carrier scaffold is a protein. A protein carrier can be modified to bind the substrate binding domain, for example through reaction of surface carboxylic acids with maleimide groups and reaction with a thiol containing substrate binding domain on the conjugate. The density of conjugates on the surface can be varied. In some embodiments, the density is determined at least partially by the amount of available functional e.g., carboxylic acid, groups on the surface.

Serum albumin, e.g., bovine serum albumin (BSA) is a commonly used carrier for immobilizing conjugated molecules to a surface. Accordingly, in some embodiments, the carrier molecule is BSA. Some commercial BSA protein carries have specific amounts of functionalization, such as 46 (average) groups per protein carrier. Therefore, in some embodiments, the maximum number of conjugates on a BSA carrier scaffold is 46.

In some embodiments, a conjugate described herein comprises: (i) a substrate binding domain; (ii) a branching domain comprising at least one of the analyte-related molecule linked to a branch point; and (iii) a linker linking the substrate binding domain and the branching domain. Each of the branching domain, linker and substrate binding domain can be the same or they can be different.

In embodiments where the device includes the immobilized conjugate and the immobilized analyte-related molecule each are comprised in a conjugate, each of the branching domain, linker-group and substrate binding domain can be the same or they can be different.

As used herein “branching domain” refers to a molecular structure that can include an inert portion and active portion. The inert portion provides a structure such as a core to which the active portion is attached external to at least a portion of the core. The branching domain can have any shape, including spherical, elliptical, a rod, a single long polymer chain, a polymer comb structure, a random coil, and have large pores (e.g., >1 nm) or include no pores or openings (e.g., <1 nm). The linker-group is also attached to the branching domain so that the branching domain can be tethered to a substrate, but where the tether can allow the branching domain to be extended away from the substrate. The terms “active” and “inert” are relative terms and depend on the branching domain environment. For example, the active portion can include functional groups, polymers or molecules that bind or interact with an antigen, molecule or polymer, while the inert portion does not directly bind or interact with the antigen, molecule or polymer. The activity can be based on the nature of the material making the inert portion and active portion, or it can be based on special considerations (e.g., accessibility to an antigen, molecule or polymer). For example, in some embodiments small molecules form at least part of the active portion of the branching domain.

In some embodiments the conjugate includes only one type of analyte-related molecule or analyte. For example, one or more, such as a plurality of small molecules having the same structure/composition, each linked to a branch point of in the linker. As used herein, a “branch-point” is an atom or molecule that can be linked to a linker and to the analyte-related molecule or analyte molecule. The analyte-related molecule or analyte molecule are linked to the branch point by a “branch” which can be a bond such as a covalent or dative bond. In some embodiments, the branch comprises at least part of the inert portion of the branching domain.

The branching domain can be represented by the formula C(x)ₐM_{b} where C is a subunit of the branching domain having a maximum possible branches equal to x. Each branch can be linked to another C, or to the analyte-related molecule M. In some embodiments, not all the branches are linked (to either C or M) and are left as open or unoccupied. The integers a and b are constrained as follows: a and b are independently integers≥1, provided that b≤(a)(x−2)+1. It is understood that if a=1, then the single subunit C is equal to the branch-point. Structure 2 shows and embodiment where x=2, a=1 and b=1. Structure 3 shows an embodiment where x=4, a=2, and b=4. Structure 4 shows an embodiment where x=3, a=5 and b=5. The “L” refers to a linker group, which is not part of the branching domain, and the unit “B” refers to an open branch point e.g., a non-occupied site not bound to M or L. Structure 2 exemplifies the simplest embodiment in that the minimum number of elements are used to construct the branching domain, with one analyte-related molecule M and one branch coupling M to the linker group L. Structure 3 exemplifies an embodiment where all the possible branch points are used for binding to either L or M. Structure 4 exemplifies an embodiment where x, the maximum possible branch points, is not used for bonding to M or L and therefore one position “B” is left open. In some embodiments more than one linker can be attached to the branching domain, while in other embodiments as shown by Structure 2, 3 and 4, only one linker is attached. In some embodiments, the inert portion of the branching domain comprises at least a portion of C and the active portion of the branching domain includes at least a portion of one or more of M. In some embodiments the branching domain can be “multivalent” with respect to the analyte-related molecule M attached to branch points of the branching domain as shown by Structures 3 and 4. As used herein multivalent refers to two or more of the small molecules in the branching domain. In some embodiments, the branching domain is “monovalent,” where only one analyte-related molecule M is attached to a branch C as shown by structure 2.

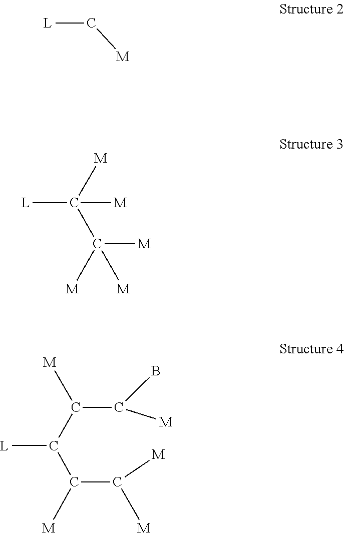

In some embodiments, C is a lysine group, wherein the branching domain comprises a first lysine linked to a second lysine, and wherein the carboxyl group of the first lysine is linked to the epsilon-amino group of second lysine. In some embodiments, the branching domain comprises a first lysine, a second lysine and a third lysine, and wherein the carboxyl group of the first lysine is linked to the epsilon-amino group of the second lysine, and the carboxyl group of the third lysine is linked to the alpha-amino group of the first or second lysine.

In some embodiments, the analyte-related molecule is selected to have the same structure as a small molecule analyte target of a method for detecting the presence of an analyte in a liquid sample as described herein. In some embodiments functional groups such as amino, carboxyl, thiol, hydroxyl that are part of the target analyte are used for forming a link to a branch in the branching domain.

In other embodiments functional groups such as amino, carboxyl, thiol, hydroxyl that are not part of the target small molecule are used for forming a link to the branchpoint in the linker.

For example, in some embodiments the analyte-related molecule or analyte molecule M can be a histadine-derived molecule, shown as Structure 5, which is linked to a branch unit C, where the analyte is histamine shown as Structure 6. For example, the branching group C can include a group that can be condensed with the carboxylate group of the histadine, such as an amine of lysine. This contrasts with embodiments wherein the histadine is linked directly, such as through the amine group as depicted in histadine-derived Structure 7. Without being bound to any specific mechanism or theory, it is proposed that by using the functional group that is not part of the target molecule, the analyte-related molecule is adapted for orienting the analyte-related molecule for binding with a capture agent capable of binding specifically with the analyte. That is, in the example of histadine derived molecule (5), the entire fragment that is the molecule histamine (6) including the amine group, can be presented to the capture agent; whereas in the histamine derived molecule (7) the amine group, which is part of the analyte to be detected, is linked directly through branching group C.

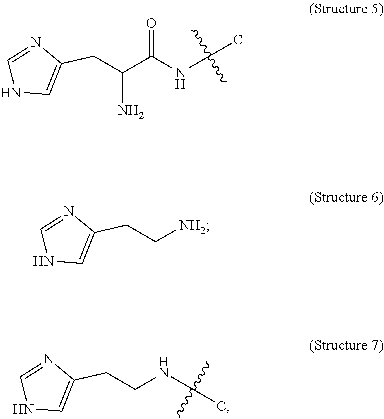 [SMILES: *NC(=O)C(N)Cc1c[nH]cn1 ; NCCc1c[nH]cn1 ; *NCCc1c[nH]cn1]

As used herein the term “linking” and “linked” refers to forming a direct or indirect attachment or connection between at least two atoms or molecules. The attachment can be by a direct chemical bond between the two atoms or molecules or by an intermediate atom or molecule. For example, F can be linked to H directly, e.g., with a covalent or other bond “-”, to form the structure “F-H” or it can be linked indirectly through G by the structure “F-G-H.” The intermediate can include, for example, an atom, a small molecule, a polymer, a protein, or a functional group.

As used herein the “substrate” can be any material comprising the sheeting or webbing material of the LFA such that it can be linked to the linker. The sheeting or webbing material can also include a coating such as a polymer or protein coating which can be functionalized, e.g., for reaction with the linker. In embodiments wherein the linker has a specified length, the length is the linear length from the head to tail group, wherein the head group is attached to the analyte-related molecule or analyte and the tail group is attached to the substrate surface. In some embodiments the length is the contour length, which is length of the linker in its maximally extended conformation and wherein none of the bonds are strained in length or angle from their lowest energy configuration. For example, where the polymer comprises a carbon or carbon oxygen chain, the eclipsed conformation is used. For example, the contour length for a single unit of a poly ethylene oxide (PEO) chain (e.g., —CH₂—CH₂—O—) has a contour length of 0.28 in water. It is understood that in addition to the molecular weight, the length of a linker will depend on the molecular dynamics, wherein, for example, the medium has a large contribution. One measurement of length that contrasts with the contour length is the Flory radius which is calculated using the random walk law, and applies, for the most part, in the melt. That is, when a polymer is put in solution with an organic solvent, the coil expands to a larger size than the size reflected by the Flory radius equation. Table 1 illustrates the contour length as compared to Flory radius for PEO

TABLE 1
PEG lengths

| Number of PEO | MW | Contour length | Flory radius |
|---|---|---|---|
| units | (Dalton) | (nm) | (nm) |
| 2 | 88 | 0.6 | 0.5 |
| 11 | 484 | 3.1 | 1.2 |
| 45 | 2000 | 12.7 | 2.8 |

In some embodiments, the structures, compositions and methods described herein can be useful for a rapid assay, for example, for testing for the presence of a small molecule. Accordingly, provided herein is a method for detecting the presence of an analyte in a liquid sample. Generally, the method comprises: (i) contacting the liquid sample to the sample receiving zone of a device described herein; and (ii) observing in the capture zones a detectable signal from a detectable attached to capture agent. The detectable signal is inversely proportional to a concentration of the analyte in the sample.

Without wishing to be bound by a theory, the capture agent comprising a detectable label binds with the analyte prior to flowing through the capture zones. The immobilize conjugate in the capture zones bind and retain any free capture agent, i.e., capture agent molecules that are not bound to the analyte. Once any free capture agent binds to the capture zones, detectable signal from the detectable label can be detected. The amount of free capture agent is inversely proportion to the amount of analyte in the sample. In other words, the assay detects the binding of the free capture agent, i.e., capture agents not bound with analyte, to the competitive molecule immobilized in the capture zones. The analyte and the immobilized molecule compete for the capture agent and can't bind to the capture agent at the same time. Thus, if there is more analyte in the sample, the signal is low since most of the capture agents already are bound with the analyte and flow through the capture zones. If there is little or no analyte in the sample, most of the capture agents remain free for binding with the competitive molecule in the capture zone. Accordingly, the detectable signal is inversely proportional to the amount of analyte in the sample.

In some embodiments, the method further comprises combining the detectable signals from two or more capture zones to provide a processed signal. In some embodiments, combining the detectable signals comprises averaging the detectable signal.

As used herein the term “rapid” refers to methods that take less time for detecting the molecule than previous comparable methods. A comparable method refers to test for the same target and providing a similar precision and sensitivity (e.g., wherein similar here means±10%). The comparable method can include the same methods and compositions as the instant test without use of the LFA described herein. For example, where a known ELISA method for detecting a small molecule in a sample takes T1 time to perform, the methods describe herein can be used to detect the small molecule in the sample in time T2, where T2 is less than T1 (e.g., with T2 is a third or less than T1, T2 half or less of T1, T2 is at least an order of magnitude less than T2). The rapidity can be, for example, determined by one rate limiting process, such as an incubation time. Without being bound by a specific theory, in some embodiments, the methods described herein can detect a small molecule more rapidly than comparative methods because the sensitivity to the small molecule is higher and less time is required for a detectable signal (e.g., above noise) to be acquired. In some embodiments, the assay can detect a small molecule through the elimination of a step used in the comparative test. For example, in a competitive ELISA assay, a test can include incubating with an analyte binding ligand to allow the competition to be established. Typically, a labeling molecule with a detectable label is then added to allow detection of the analyte binding ligand. By conjugating a detectable label to the capture agent, the step of adding the labeling molecule is eliminated. In addition, using more than one capture zones 62 for detection can provide for more accuracy and lower detection limits. In some embodiments the compositions and structures described herein can be used for the detection of a small molecule in less than one hour, e.g., less than 40 min, less than 20 min, less than 10 min or even less than 5 min.

In some embodiments the assay can detect a concentration of less than 500 nM of the target analyte (e.g., histamine). In some embodiments the assay can detect less than 400 nM of the target analyte e.g., less than 300, less than 200, less than 100, less than 50 nM, less than 10 nM or even less than 5 nM. In some embodiments the assay can detect the target analyte in the range between about 1 and 500 nM, such as between about 5 and 100 nM.

In some embodiments the assay for a small molecule (e.g. a histamine, cortisol or a DNP assay) includes the immobilization of conjugates to the solid support. In some embodiments the assay includes the immobilization of the detecting molecule (e.g., anti-histamine antibody, or anti-DNP antibody) on the solid support.

### Test Sample

In accordance with various embodiments described herein, a test sample, including any fluid or specimen (processed or unprocessed) that is intended to be evaluated for the presence of a small molecule can be subjected to methods, compositions, kits and systems described herein. The test sample or fluid can be liquid, supercritical fluid, solutions, suspensions, gases, gels, slurries, and combinations thereof. The test sample or fluid can be aqueous or non-aqueous.

In some embodiments, the test sample can be an aqueous fluid. As used herein, the term “aqueous fluid” refers to any flowable water-containing material that is suspected of comprising an analyte such as a target small molecule.

In some embodiments, the test sample can include a biological fluid obtained from a subject. Exemplary biological fluids obtained from a subject can include, but are not limited to, blood (including whole blood, plasma, cord blood and serum), lactation products (e.g., milk), amniotic fluids, sputum, saliva, urine, semen, cerebrospinal fluid, bronchial aspirate, perspiration, mucus, liquefied stool sample, synovial fluid, lymphatic fluid, tears, tracheal aspirate, and any mixtures thereof. In some embodiments, a biological fluid can include a homogenate of a tissue specimen (e.g., biopsy) from a subject. In one embodiment, a test sample can comprise a suspension obtained from homogenization of a solid sample obtained from a solid organ or a fragment thereof.

In some embodiments, the test sample can include a fluid or specimen obtained from an environmental source. For example, the fluid or specimen obtained from the environmental source can be obtained or derived from food products or industrial food products, food produce, poultry, meat, fish, beverages, dairy products, water (including wastewater), surfaces, ponds, rivers, reservoirs, swimming pools, soils, food processing and/or packaging plants, agricultural places, hydrocultures (including hydroponic food farms), pharmaceutical manufacturing plants, animal colony facilities, and any combinations thereof.

In some embodiments, the test sample can include a fluid or specimen collected or derived from a biological culture. For example, a biological culture can be a cell culture. Examples of a fluid or specimen collected or derived from a biological culture includes the one obtained from culturing or fermentation, for example, of single- or multi-cell organisms, including prokaryotes (e.g., bacteria) and eukaryotes (e.g., animal cells, plant cells, yeasts, fungi), and including fractions thereof. In some embodiments, the test sample can include a fluid from a blood culture. In some embodiments, the culture medium can be obtained from any source, e.g., without limitations, research laboratories, pharmaceutical manufacturing plants, hydrocultures (e.g., hydroponic food farms), diagnostic testing facilities, clinical settings, and any combinations thereof.

In some embodiments, the test sample can include a media or reagent solution used in a laboratory or clinical setting, such as for biomedical and molecular biology applications. As used herein, the term “media” refers to a medium for maintaining a tissue, an organism, or a cell population, or refers to a medium for culturing a tissue, an organism, or a cell population, which contains nutrients that maintain viability of the tissue, organism, or cell population, and support proliferation and growth.

In some embodiments, the test sample can be a non-biological fluid. As used herein, the term “non-biological fluid” refers to any fluid that is not a biological fluid as the term is defined herein. Exemplary non-biological fluids include, but are not limited to, water, salt water, brine, buffered solutions, saline solutions, sugar solutions, carbohydrate solutions, lipid solutions, nucleic acid solutions, hydrocarbons (e.g. liquid hydrocarbons), acids, gasolines, petroleum, liquefied samples (e.g., liquefied samples), and mixtures thereof

It can be necessary or desired that a test sample, such be preprocessed prior to small molecule detection as described herein, e.g., with a preprocessing reagent. Even in cases where pretreatment is not necessary, preprocess optionally can be done for mere convenience (e.g., as part of a regimen on a commercial platform). A preprocessing reagent can be any reagent appropriate for use with the methods described herein.

The sample preprocessing step generally comprises adding one or more reagents to the sample. This preprocessing can serve a number of different purposes, including, but not limited to, hemolyzing cells such as blood cells, dilution of sample, etc. The preprocessing reagents can be present in the sample container before sample is added to the sample container or the preprocessing reagents can be added to a sample already present in the sample container. When the sample is a biological fluid, the sample container can be a VACUTAINER®, e.g., a heparinized VACUTAINER®.

The preprocessing reagents include, but are not limited to, surfactants and detergents, salts, cell lysing reagents, anticoagulants, degradative enzymes (e.g., proteases, lipases, nucleases, lipase, collagenase, cellulases, amylases and the like), and solvents, such as buffer solutions.

After the optional preprocessing step, the sample can be optionally further processed by adding one or more processing reagents to the sample. These processing reagents can degrade unwanted molecules present in the sample and/or dilute the sample for further processing. These processing reagents include, but are not limited to, surfactants and detergents, salts, cell lysing reagents, anticoagulants, degradative enzymes (e.g., proteases, lipases, nucleases, lipase, collagenase, cellulases, amylases, heparanases, and the like), and solvents, such as buffer solutions. Amount of the processing reagent to be added can depend on the particular sample to be analyzed, the time required for the sample analysis, identity of the small molecule to be detected or the amount of small molecule present in the sample to be analyzed.

It is not necessary, but if one or more reagents are to be added they can present in a mixture (e.g., in a solution, “processing buffer”) in the appropriate concentrations. Amount of the various components of the processing buffer can vary depending upon the sample, small molecule to be detected, concentration of the small molecule in the sample, or time limitation for analysis.

Reagents and treatments for processing blood before assaying are also well known in the art, e.g., as used for assays on Abbott TDx, AxSYM®, and ARCHITECT® analyzers (Abbott Laboratories), as described in the literature (see, e.g., Yatscoff et al., Abbott TDx Monoclonal Antibody Assay Evaluated for Measuring Cyclosporine in Whole Blood, Clin. Chem. 36: 1969-1973 (1990), and Wallemacq et al., Evaluation of the New AxSYM Cyclosporine Assay: Comparison with TDx Monoclonal Whole Blood and EMIT Cyclosporine Assays, Clin. Chem. 45: 432-435 (1999)), and/or as commercially available. Additionally, pretreatment can be done as described in U.S. Pat. No. 5,135,875, European Pat. Pub. No. 0 471 293, U.S. Provisional Pat. App. 60/878,017, filed Dec. 29, 2006, and U.S. Pat. App. Pub. No. 2008/0020401, content of all of which is incorporated herein by reference. It is to be understood that one or more of these known reagents and/or treatments can be used in addition to or alternatively to the sample treatment described herein.

After addition of the processing reagents, the sample can be incubated for a period of time, e.g., for at least one minute, at least two minutes, at least three minutes, at least four minutes, at least five minutes, at least ten minutes, at least fifteen minutes, at least thirty minutes, at least forty-five minutes, or at least one hour. Such incubation can be at any appropriate temperature, e.g., room-temperature (e.g., about 16° C. to about 30° C.), a cold temperature (e.g. about 0° C. to about 16° C.), or an elevated temperature (e.g., about 30° C. to about 95° C.). In some embodiments, the sample is incubated for less than about 10 minutes at room temperature (e.g., less than about 8 minutes, less than about 5 minutes).

### Kits

A kit comprising at least one composition described herein is also provided.

Some embodiments the kit comprises a LFA, such as an LFA enclosed in a housing, and any one or more of preprocessing regents, informational material, containers and a carrier.

The kits can include, but are not limited to, any of the preprocessing reagents as described herein.

In some embodiments, the informational material can be descriptive, instructional, marketing or other material that relates to the methods described herein and/or the use of the aggregates for the methods described herein. For example, the informational material can describe methods for using the kits provided herein to perform an assay for detection of a target entity, e.g., a small molecule. The kit can also include an empty container and/or a delivery device, e.g., which can be used to deliver or prepare a test sample to a test container.

The informational material of the kits is not limited in its form. In many cases, the informational material, e.g., instructions, is provided in printed matter, e.g., a printed text, drawing, and/or photograph, e.g., a label or printed sheet. However, the informational material can also be provided in other formats, such as Braille, computer readable material, video recording, or audio recording. In another embodiment, the informational material of the kit is a link or contact information, e.g., a physical address, email address, hyperlink, website, or telephone number, where a user of the kit can obtain substantive information about the formulation and/or its use in the methods described herein. Of course, the informational material can also be provided in any combination of formats.

In some embodiments, the kit can contain separate containers, dividers or compartments for each component and informational material. For example, each different component can be contained in a bottle, vial, or syringe, and the informational material can be contained in a plastic sleeve or packet. In other embodiments, the separate elements of the kit are contained within a single, undivided container. For example, a collection of the magnetic particles is contained in a bottle, vial or syringe that has attached thereto the informational material in the form of a label.

In some embodiments the kit includes a carrier for organizing and protecting the components in the kit during transport or storage. The carrier can be in any form including a bag, a box or a case, including handles, straps and wheels for convenient movement or storage.

Some exemplary embodiments can be described as follows:

Embodiment 1: A lateral flow assay device for detecting the presence of a small molecule analyte in a liquid sample, comprising: a lateral flow matrix which defines a flow path and which comprises in series: (i) a sample receiving zone; and (ii) one or more capture zones, wherein each capture zone independently comprises a conjugate immobilized on the lateral flow matrix, wherein the conjugate comprises one or more analyte-related molecules conjugated to the lateral flow matrix via a linker and wherein the linker is not a protein, wherein the conjugate is adapted for orienting the analyte-related molecule for binding with a capture agent capable of binding specifically with the analyte, and wherein the analyte-related molecule and the analyte competitively bind with said capture agent.

Embodiment 2: The device of Embodiment 1, wherein the linker has a length between 5 and 200 angstroms.

Embodiment 3: The device of Embodiment 1 or 2, wherein the linker comprises a polyethylene glycol (PEG).

Embodiment 4: The device of any one of Embodiments 1-3, wherein the linker comprises at least one lysine.

Embodiment 5: The device of Embodiment 4, wherein at least one analyte-related molecule is linked to the alpha-amino group of the at least one lysine and at least one analyte-related molecule is linked to the epsilon-amino group of the at least one lysine.

Embodiment 6: The device of any one of Embodiments 1-5, wherein the linker comprises a first lysine linked to a second lysine, and wherein the carboxyl group of the first lysine is linked to the epsilon-amino group of second lysine.

Embodiment 7: The device of any one of Embodiments 1-6, wherein the linker comprises a first lysine, a second lysine and a third lysine, and wherein the carboxyl group of the first lysine is linked to the epsilon-amino group of the second lysine, and the carboxyl group of the third lysine is linked to the alpha-amino group of the first or second lysine.

Embodiment 8: The device of any one of Embodiments 1-7, wherein the capture agent is an antibody.

Embodiment 9: The device of any one of Embodiments 1-8, wherein the capture agent comprises a detectable label.

Embodiment 10: The device of any one of Embodiments 1-9, wherein the analyte is selected from the group consisting of amino acids, nucleosides, saccharides, steroids, hormones, therapeutic agents, metabolites of therapeutic agents.

Embodiment 11: The device of any one of Embodiments 1-10, wherein the analyte is histamine.

Embodiment 12: The device of any one of Embodiments 1-11, wherein the analyte-related molecule comprises an imidazole group.

Embodiment 13: The device of Embodiment 12, wherein the analyte-related molecule is histadine.

Embodiment 14: The device of any one of Embodiments 1-13 wherein the device comprises a plurality of serially oriented capture zones.

Embodiment 15: The device of any of Embodiments 1-14, wherein an amount of the immobilized conjugate in at least two capture zones is different.

Embodiment 16: The device of any one of Embodiments 1-15, wherein an amount of the immobilized conjugate in a capture zone closer to the sample receiving zone is lower than an amount of the immobilized conjugate in a capture zone further from the sample receiving zone.

Embodiment 17: The device of any one of Embodiments 1-16, wherein an amount of the immobilized conjugate in each capture zone is lower than an amount of the immobilized conjugate in each capture zone that is further from the sample receiving zone.

Embodiment 18: The device of any one of Embodiments 1-17, further comprising a first control zone, wherein the first control zone comprises an analyte molecule immobilized on the lateral flow matrix.

Embodiment 19: The device of Embodiment 18, where the first control zone comprises a BSA conjugated to the analyte molecule.

Embodiment 20: The device of Embodiment 18 or 19, wherein the analyte molecule is histamine.

Embodiment 21: The device of any one of Embodiments 18-20, wherein the first control zone is positioned next to a capture zone so that the distance from the sample receiving zone to the first control zone and the distance from the sample receiving zone to the capture zone are substantially equal.

Embodiment 22: The device of any one of Embodiments 1-21, further comprising a second control zone comprising an anti-Fc capture agent.

Embodiment 23: The device of Embodiment 22, wherein the second control zone is positioned in series after the capture zones, wherein a liquid flowing from the sample zone reaches the capture zones before reaching the second control zone.

Embodiment 24: The device of any one of Embodiments 1-23, wherein the sample receiving zone comprises: (i) a labeling zone comprising a diffusively bound capture agent; and (ii) a sample zone for receiving a liquid sample comprising the analyte.

Embodiment 25: The device of Embodiment 24, wherein the labeling zone is positioned between the plurality of capture zones and the sample zone for receiving the liquid sample comprising the analyte.

Embodiment 26: The device of any one of Embodiments 1-25, wherein each capture zone independently has a regular or irregular shape.

Embodiment 27: The device of any one of Embodiments 1-26, wherein at least one of the capture zone has a shape selected from the group consisting of a line, a circle, a rod, and a polygonal.

Embodiment 28: The device of Embodiment 27, wherein said polygonal is a square, a triangle or a rectangle.

Embodiment 29: The device of any one of Embodiments 1-28, wherein at least two capture zones are the same shape.

Embodiment 30: The device of any one of Embodiments 1-29, wherein at least two capture zones are of same size.

Embodiment 31: The device of any one of Embodiments 1-30, wherein a binding affinity of the analyte binding with the capture agent is higher than a binding affinity of the immobilized conjugate binding with the capture agent.

Embodiment 32: A method for detecting the presence of an analyte in a liquid sample, the method comprising: (i) contacting the liquid sample to the sample receiving zone of the device of any one of Embodiments 1-31, wherein the capture agent comprises a detectable label; and (ii) observing a detectable signal from the detectable label in the capture zones, wherein the detectable signal is inversely proportional to a concentration of the analyte in the sample.

Embodiment 33: The method according to Embodiment 32, further comprising combining the detectable signals from two or more capture zones to provide a processed signal.

Embodiment 34: The method according to Embodiment 33, wherein the detectable signal is provided as a quantified value and combining comprises summing the detectable signals from said two or more capture zones to provide the processed signal.

Embodiment 35: The method according to Embodiment 33, wherein the detectable signal is provided as a quantified value and said combining the detectable signals comprises averaging the detectable signal from said two or more capture zones to provide the processed signal.

Embodiment 36: A device for detecting the presence of a small molecule analyte in a liquid sample, comprising: a lateral flow matrix which defines a flow path and which comprises in series: (i) a sample receiving zone; and (ii) one or more capture zones, wherein each capture zone independently comprises a conjugate immobilized on the lateral flow matrix, and an amount of the immobilized conjugate in a capture zone closer to the sample receiving zone is lower than an amount of the immobilized conjugate in a capture zone further from the sample receiving zone.

Embodiment 37: The device according to Embodiment 36, wherein the conjugate comprises one or more analyte-related molecules conjugated to the lateral flow matrix via a linker and wherein the linker is not a protein, and wherein the conjugate is adapted for orienting the analyte-related molecule for binding with a capture agent capable of binding specifically with the analyte.

Embodiment 38: The device according to Embodiment 36 or 37, wherein an amount of the immobilized conjugate in each capture zone is lower than an amount of the immobilized conjugate in each capture zone that is further from the sample receiving zone.

### Some Selected Definitions

As used in this specification and the appended claims, the singular forms “a,” “an,” and “the” include plural references unless the context clearly dictates otherwise. Thus for example, references to “the method” includes one or more methods, and/or steps of the type described herein and/or which will become apparent to those persons skilled in the art upon reading this disclosure and so forth. Similarly, the word “or” is intended to include “and” unless the context clearly indicates otherwise.

Other than in the operating examples, or where otherwise indicated, all numbers expressing quantities of ingredients or reaction conditions used herein should be understood as modified in all instances by the term “about.” The term “about” when used to described the present invention, in connection with percentages means±1%.

In one aspect, the present invention relates to the herein described compositions, methods, and respective component(s) thereof, as essential to the invention, yet open to the inclusion of unspecified elements, essential or not (“comprising”). In some embodiments, other elements to be included in the description of the composition, method or respective component thereof are limited to those that do not materially affect the basic and novel characteristic(s) of the invention (“consisting essentially of”). This applies equally to steps within a described method as well as compositions and components therein. In other embodiments, the inventions, compositions, methods, and respective components thereof, described herein are intended to be exclusive of any element not deemed an essential element to the component, composition or method (“consisting of”).

It should be understood that this invention is not limited to the particular methodology, protocols, and reagents, etc., described herein and as such may vary. The terminology used herein is for the purpose of describing particular embodiments only, and is not intended to limit the scope of the present invention, which is defined solely by the claims.

All patents, patent applications, and publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these

### EXAMPLES

The following examples illustrate some embodiments and aspects of the invention. It will be apparent to those skilled in the relevant art that various modifications, additions, substitutions, and the like can be performed without altering the spirit or scope of the invention, and such modifications and variations are encompassed within the scope of the invention as defined in the claims which follow. The following examples do not in any way limit the invention.

### Example: 1

The examples disclose a rapid immunochromatographic, LFA test for small molecule detection. As a case study, rapid detection of histamine is presented as an early anaphylactic shock biosensor through the detection of rises in histamine concentration over the nano molar range. The assay disclosed is based on a competitive immunoassay that uses BSA-histidine conjugates immobilized on nitrocellulose along with histamine-binding antibodies attached to gold nanoparticles (FIG. 1). The antibodies recognize the BSA-histidine conjugates and bind to them, forming a red spot as a result of gold nanoparticle accumulation. If histamine is present in the solution, it binds to the antibody and blocks the antibody from binding to the BSA-histidine; resulting in a lower signal. Increasing histamine concentrations lead to decreased signals in the immunoassay.

The detection device is based on immunochromatographic nitrocellulose strips (both LFA and half-strip assays. Typical competitive LFAs consist of paper strips to which a biological sample is added, and the fluid wicks through, resulting in two or more colored spots for a negative test, or only one spot for a positive test. A device that uses a simplified format of the LFAs is the half-strip assay, where the conjugate pad (e.g., a conjugate zone 40) and sample pad 20 are substituted by a solution into which the nitrocellulose with the absorbent pad is immersed. This eliminates the need to dry down the NP-antibody conjugate. In some examples, test bands/spots 62 are spotted with either BSA-histamine conjugate or BSA-histidine conjugates with different histidine linkers (mono or dual histidine). Different concentrations of the test spot were also studied. FIG. 1 shows an example of histamine competitive assay strip with an anti-Fc positive control 64 (anti-Fc), and two BSA-histidine regions 62 (BSA-histidine) (from left to right in FIG. 1). The conjugate includes an anti-histamine antibody (MAA927Ge21, Cloud-Clone) conjugated to gold nanoparticles (NPs).

Synthesis of antibody conjugated to gold nanoparticles: First, gold NPs were synthesized and conjugated to antibodies by either covalent attachment or passive adsorption. Briefly, 18 nm diameter gold nanoparticles were synthesized by adding 1 ml of a 6.8 mM sodium citrate solution to 50 ml of 0.25 mM gold (III) chloride, while the gold chloride solution is boiling. Samples were stirred and heated for 15 min during which the gold crystals form. Once the solution reached room temperature, ˜0.5 mg of bis(P-sulfonatophenyl) dihydrate dipotassium salt was added and the solution, and mixing continues overnight. Samples were left to cool down to room temperature while stirring continues.

Prior to antibody conjugation, the NPs were separated from excess reagents by centrifugation at 12000 rcf for 12 min. The resulting NP pellet was resuspended in 100 l of 40 mM HEPES at pH 7.7 and 300 μl of MilliQ water, followed by the addition of 2.5 μl of 1 mg/ml antibody, vortexed, and further agitated overnight, to enable antibody binding to the NP. In order to avoid nonspecific binding on the NP, 5 l of 0.1 mM mPEG was added, the solution was vortexed and further agitated for 20 min, to enable mPEG to passivate any bare gold surfaces. Finally, NPs were centrifuged for 15 min at 12000 rcf to separate excess reagents. The AuNPs were diluted to an absorbance of 0.68 AU at 520 nm, and used in the immunoassays.

Synthesis of Histamine conjugates: This study used BSA as a carrier protein (scaffold) to immobilize histidine linkers. For the study, maleimide modified BSA was used to link thiol-modified PEG-Mono-Histidine (Structure 1, previously shown). Briefly, thiol-PEG-mono-histidine was synthesized on the Rink Amide LL resin (1111 mg). A 1-(4,4-dimethyl-2,6-dioxocyclohex-1-ylidene)isovaleryl (ivDde) protected Fmoc-Lys amino acid (1 mmole) was first attached to the Rink Amide LL resin using 2-(1H-benzotriazole-1-yl)-1,1,3,3-tetramethyluronium hexafluorophosphate (HBTU) (0.9 mmole) and N,N-diisopropylethylamine (DIEA, 100 μL). The reaction was allowed to proceed for 2 h. Thereafter, the fluorenylmethyloxycarbonyl (Fmoc) group of the lysine was removed and HBTU activated Thiol-EG6-COOH (0.72 mmole) was added to the resin and allowed to react for 1 h at room temperature while agitating. Next, the ivDde group on the Lys was removed using 2% hydrazine solution and the Fmoc-protected histidine amino acid (1 mmole) was reacted with the linker on the resin. After 1 h of reaction, the Fmoc group of the histidine was cleaved by using 20% piperidine in dimethylformamide (DMF), and synthesized linker was removed from the resin using trifluoroacetic acid (TFA) cleavage cocktail (92.5% TFA, 2.5% H₂O, 2.5% TIS, 2.5% EDT), and collected. All organic solvents were removed before purification of the molecule on the C18 column using an RP-HPLC system. Thiol-PEG-mono-Histidine linker was characterized using LC-MS, and expected mass was 634.34; found 635.3. The purity of the synthesized molecule was >95%.

Subsequently, 1 mL of maleimide conjugation buffer obtained from Thermo scientific was added to 10 mg of purified thiol-PEG-mono Histidine linker. Following this, the 1 mL solution of thiol-PEG-mono Histidine was added to 5 mg of commercially available BSA-maleimide substrate and allowed to react at room temperature for 4 h. Finally, the histidine conjugated BSA was purified using a 10K spin column. BSA dual-histidine is synthesized using the same protocol. BSA-histamine (Product: 80-1460) is purchased from Fitzgerald.

Antibody application to nitrocellulose membranes: Optimized nitrocellulose membrane (Sartorius, CN95) was cut into strips using a laser cutter (Universal Laser Systems). For the positive control area, 0.5 μl of anti-mouse Fc antibody (1 mg/ml; AQ127, EMDMillipore) was spotted on the control line. The BSA-histamine capture line on the nitrocellulose was generated by pipetting 0.5 μl of BSA-histidine (at varying concentrations 0.25-0.5 mg/ml) at the test areas. Strips are air-dried and stored in a desiccator at room temperature before use. (Note: For half-strip assays, 0.4 μl BSA-histidine is used).

Table 2 lists some components of the LFA. The components of LFA (sample pad, conjugate pad, nitrocellulose membrane, absorbent pad) were all optimized based on the larger significant difference of the intensity of the test spot (BSA-histidine) in the “+/−” tests (“+” test=250 nM histamine in sample, “−” test=no histamine) using Image J software.

TABLE 2
Exemplary LFA compnents (optimized component indicated denoted by *)

| LFA | Material | | | | | | | | | |
|---|---|---|---|---|---|---|---|---|---|---|
| Componnets | tested | | | | | | | | | |
| Backing | 0.10 | 0.15* | 0.20 | | | | | | | |
| Membrane | CN95L* | CN95M | CN95R | CN104L | HF75 | FF80 | HF180 | HF75 | FF120 | Prima |
| (Sartorius) | | | | | | | | | | 40 |
| Conjugate pad | 6613* | 6612 | 6615* | Standard | Standard 14 | | | | | |
| (DCN | | | | 17 | | | | | | |
| Ahistrom) | | | | | | | | | | |
| Sample pad | 1160 | 8964 | Fusioin 5* | Blooed | | | | | | |
| (Whatman/GE) | | | | separator | | | | | | |
| Nanoparticles | Laboratory | Laboratory | Nanostar | Novus | | | | | | |
| (NPs) | synthesized | synthesized | gold NPs | Biological | | | | | | |
| | 20 nm gold | 40 nm gold | | NPs | | | | | | |
| | NPs* | NPs | | | | | | | | |
| Sample | Serum* | Plasma | | | | | | | | |
| Tween | Tween 80 | | | | | | | | | |
| | purified* | | | | | | | | | |
| AB spotting. | AB | BSA- | | | | | | | | |
| BSA-histadine | | histadine* | | | | | | | | |
| spotting | | | | | | | | | | |

Immunochromatography: In a competitive assay, the target-conjugate plays a crucial role in order to obtain the desired sensitivity. In order to prove the importance of the conjugate molecule, BSA-histamine conjugate was used as a comparison to the designer BSA-histidine conjugate. Histamine concentrations can be quantified by finely tuning the affinity of the antibodies to the BSA-histidine conjugates. Histidine, Structure 5, only differs from histamine, Structure 6, by the presence of one additional carboxylate group at its end, and when it terminal amine group becomes covalently linked to the linker, it effectively exposes the remaining portion of the structure that is equivalent to the entire histamine molecule. This contrasts to linking histamine itself directly to a linker or conjugate through its amine group, which only exposes a portion of the histamine. The additional carboxylate group of histidine can be easily conjugated to a linker, such as a polyethylene glycol (PEG) polymer chain, and this results in a conjugate that mimics free histamine better as both the imidazole ring and the amine group are now available for the antibody to bind.

Each immunochromatography strip was run individually. The rapid test solution contains (i) 30 μl of human serum spiked with increasing concentrations of the histamine serum sample, 2.5 μl of 0.68 a.u. antibody-conjugated gold NPs and (iv) 8 μl of 1% tween in PBS and 4 μl of 50% sucrose in water. The run time for optimization is less than 15 min. The strips are left to dry and then imaged for quantitative signal analysis.

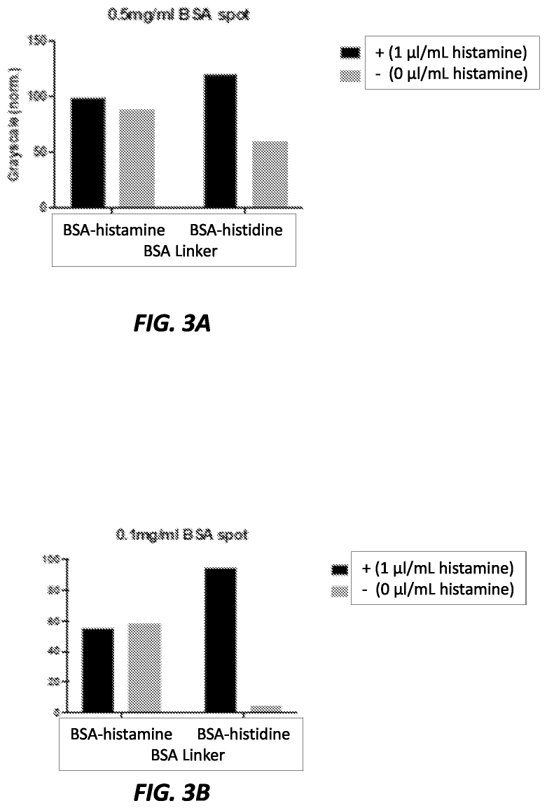

During initial experiments it was quickly realized that the antibody exhibited stronger affinities towards the histamine-BSA conjugates than to the free histamine molecule that we were interested in quantifying. As a result, when such antibodies are utilized in the development of competitive immunoassays, they failed to exhibit specific binding to free histamine. As seen in the FIGS. 3A and 3B, when using BSA-histamine as the detection biomolecule, there is negligible change after the addition of free histamine at two different concentrations (0.5 mg/mL in 3A and 0.1 mg/mL in 3B). This results from antibodies having a higher affinity for BSA-histamine than for free histamine, thus no competition with free histamine is observable. On the other hand, BSA-histidine-based tests demonstrate a significant decrease in signal in the presence of histamine in the sample, as free histamine can effectively compete with the BSA-histidine immobilized on the nitrocellulose.

The assay was further optimized by including an additional resolution factor. Such an effect was realized by adding multiple test spots. The increased resolution was achieved by leveraging the spot position and introducing a gradient that could resolve the high binding antibodies on the nitrocellulose membrane resulting in a competitive effect with free histamine in the spots closer to the sample followed by the more significant inhibition effect in the later flow towards the end of the nitrocellulose strip. Both the addition of multiple BSA-Histidine spots of different concentrations and the addition of different histidine terminations enable quantifiable detection of low nano-molar amounts of histamine.

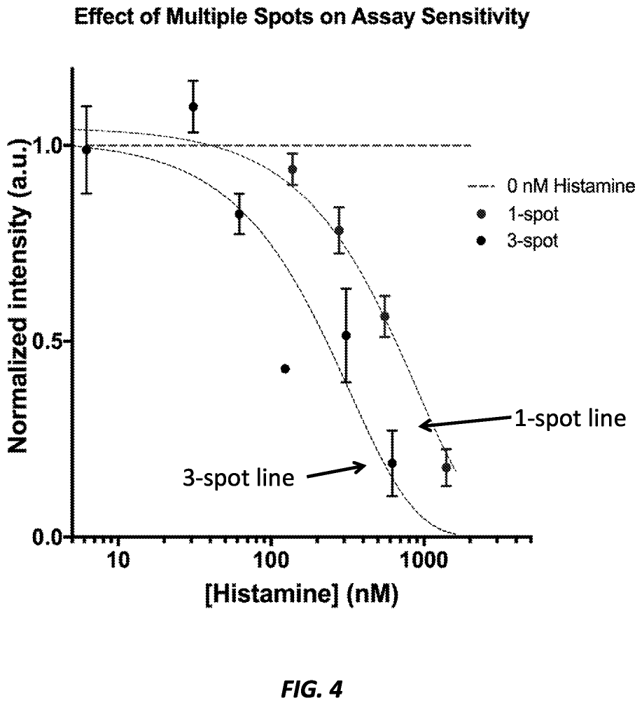

FIG. 4 demonstrates the effect of multiple spots on the improvement of sensor's sensitivity to the target. By comparing the results of 1 spot versus 3, it is observed, for instance, a 6% signal difference with 138 nM histamine with 1 spot as compared to 57% difference with 3 spots. The data points were fitted to an exponential decay equation (y=a*exp(−b*x)), where b is the decay rate. Since the data was normalized for a better comparison, the values did not differ significantly (For one spot, a=1.047; For three spots, a=1.015). However, there was a nearly three times difference in the b values (For one spot, b=0.001; For three spots, a=0.003) showing a better sensitivity with three spots, and more meaningful measurements at the concentrations of interest (between 0-200 nM).

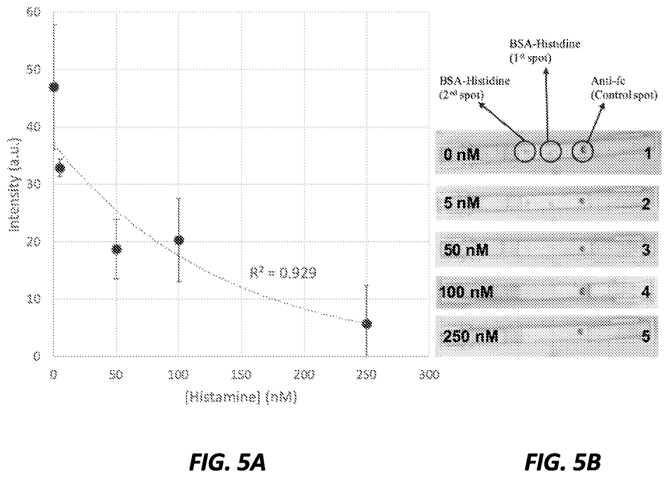

A calibration curve, FIG. 5A, was also made using a LFA including all components, depicted as strips 1 through 5 in FIG. 5B. Control detection results, showing chromatography with human serum only, are shown in the top strip (0 nM). The results show a strong signal in the positive control (anti-Fc), as well as in both BSA-histidine regions. Strip 5 is chromatographed with human serum spiked with 250 nM histamine. The result shows that only the positive control anti-Fc antibody is detected. Strips 2-4 show increasing concentrations of histamine concentrations at 5, 50 and 100 nM which are all within the clinically relevant range. Visual inspection allows for distinguishing the three clinically relevant concentrations. Rapid test results are analyzed using the image processing software ImageJ. Image analysis allows for the quantification of clinically-relevant histamine concentrations.

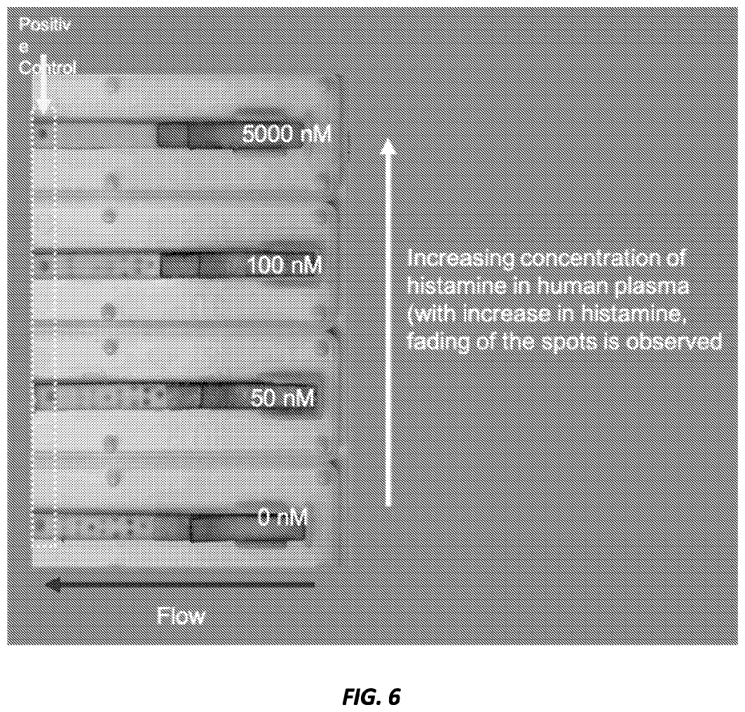

Exemplary variations: The assay is described in the form of a rapid (<15 min) assay to detect a small molecule like histamine using the paper-based immuno-chromatography approach with either a single target spot or a pattern of spots. The number of spots can be varied to obtain the desired detection range, for example as depicted in FIG. 6. Each spots also can be broken into an array of smaller spots or lines to further increase sensitivity of the assay.

In another variation an LFA using a control spot including a histamine (Structure 6) conjugate and a detection spots using histidine (structure 5) conjugates can be made. The control spot is placed next to the first detection spot to encounter the fluid sample when it is being used so that it does not interfere with the detection spots, e.g. by reducing the concentration of the analyte the control spots encounter. Because of the histamine conjugate has a high affinity to anti-histamine antibodies in the sample when present, it serves as a control as to the efficacy of the test. For example, where the competitive agent is an anti-histamine conjugated to a gold nanoparticle, the histamine conjugate (e.g., histamine-BSA) will interact strongly with the competitive agent and provide a spot where the intensity is indicative of the state of the LFA components and reagents used in the assay. This contrasts with a typical control spot such as the Anti-fc control spots depicted in FIGS. 5B and 6, which are not selective and will indicate a positive result even if an anti-histamine antibody, such as the anti-histamine antibody attached to a gold nanoparticle, is degraded or not functioning as intended.

In addition to being used as a control spot, the histamine conjugate control spot can serve as a baseline. This can be used to ratio the signals from the detection spots so that the relative intensities can be determined providing more consistent results independent of any slight degradations or conditions between tests or batches of LFA that might be used.

### REFERENCES

1. Anaphylaxis and Hypersensitivity Reactions, edited by Mariana C. Castells, Humana Press, 2010. ProQuest Ebook Central, http://ebookcentral.proquest.com/lib/harvardebooks/detail.action?docID=666901. Created from harvard-ebooks on 2019 3 Nov. 19:47:33.
2. Laroche Dl, Gomis P, Gallimidi E, Malinovsky J M, Mertes PMAnesthesiology. 2014. Diagnostic value of histamine and tryptase concentrations in severe anaphylaxis with shock or cardiac arrest during anesthesia. 121(2):272-9.
3. Buckler, R. T., Dailey, F. A., Ficalora, J. A., Gavin, J. J. and Plunkett, G. A., Bayer Corp, 1992. Histamine derivatives, immunogen conjugates and antibodies raised thereto. U.S. Pat. No. 5,112,738.
4. Fu, E.; Liang, T.; Houghtaling, J.; Ramachandran, S.; Ramsey, S. A.; Lutz, B.; Yager, P., 2011. Enhanced Sensitivity of Lateral Flow Tests Using a Two-Dimensional Paper Network Format. Analytical Chemistry. 83 (20), 7941-7946.

What is claimed is:

1. A lateral flow assay device for detecting the presence of a small molecule analyte in a liquid sample, comprising:
a lateral flow matrix which defines a flow path and which comprises in series:
a sample receiving zone; and
one or more capture zones,
wherein each capture zone independently comprises a conjugate immobilized on the lateral flow matrix,
wherein the conjugate comprises one or more analyte-related molecules conjugated to the lateral flow matrix via a linker and wherein the linker is not a protein,
wherein the conjugate is adapted for orienting the analyte-related molecule for binding with a capture agent capable of binding specifically with the analyte, and
wherein the analyte-related molecule and the analyte competitively bind with said capture agent.

2. The device of claim 1, wherein the linker has a length between 5 and 200 angstroms.

3. The device of claim 1, wherein the linker comprises a polyethylene glycol (PEG).

4. The device of claim 1, wherein the linker comprises at least one lysine.

5. The device of claim 4, wherein at least one analyte-related molecule is linked to the alpha-amino group of the at least one lysine and at least one analyte-related molecule is linked to the epsilon-amino group of the at least one lysine.

6. The device of claim 1, wherein the linker comprises a first lysine linked to a second lysine, and wherein the carboxyl group of the first lysine is linked to the epsilon-amino group of second lysine.

7. The device of claim 1, wherein the linker comprises a first lysine, a second lysine and a third lysine, and wherein the carboxyl group of the first lysine is linked to the epsilon-amino group of the second lysine, and the carboxyl group of the third lysine is linked to the alpha-amino group of the first or second lysine.

8. The device of claim 1, wherein the capture agent is an antibody.

9. The device of claim 1, wherein the capture agent comprises a detectable label.

10. The device of claim 1, wherein the analyte is selected from the group consisting of amino acids, nucleosides, saccharides, steroids, hormones, therapeutic agents, metabolites of therapeutic agents.

11. The device of claim 10, wherein the analyte is histamine.

12. The device of claim 1, wherein the analyte-related molecule comprises an imidazole group.

13. The device of claim 12, wherein the analyte-related molecule is histadine.

14. The device of claim 1, wherein the device comprises a plurality of serially oriented capture zones.

15. The device of claim 14, wherein an amount of the immobilized conjugate in at least two capture zones is different.

16. The device of claim 15, wherein an amount of the immobilized conjugate in a capture zone closer to the sample receiving zone is lower than an amount of the immobilized conjugate in a capture zone further from the sample receiving zone.

17. The device of claim 16, wherein an amount of the immobilized conjugate in each capture zone is lower than an amount of the immobilized conjugate in each capture zone that is further from the sample receiving zone.

18. The device of claim 1, further comprising a first control zone, wherein the first control zone comprises an analyte molecule immobilized on the lateral flow matrix.

19. The device of claim 18, where the first control zone comprises a BSA conjugated to the analyte molecule.

20. The device of claim 18, wherein the analyte molecule is histamine.

21. The device of claim 18, wherein the first control zone is positioned next to a capture zone so that the distance from the sample receiving zone to the first control zone and the distance from the sample receiving zone to the capture zone are substantially equal.

22. The device of claim 18, further comprising a second control zone comprising an anti-Fc capture agent.

23. The device of claim 22, wherein the second control zone is positioned in series after the capture zones, wherein a liquid flowing from the sample zone reaches the capture zones before reaching the second control zone.

24. The device of claim 1, wherein the sample receiving zone comprises: (i) a labeling zone comprising a diffusively bound capture agent; and (ii) a sample zone for receiving a liquid sample comprising the analyte.

25. The device of claim 24, wherein the labeling zone is positioned between the plurality of capture zones and the sample zone for receiving the liquid sample comprising the analyte.

26. The device of claim 1, wherein each capture zone independently has a regular or irregular shape.

27. The device of claim 1, wherein at least one of the capture zone has a shape selected from the group consisting of a line, a circle, a rod, and a polygonal.

28. The device of claim 27, wherein said polygonal is a square, a triangle or a rectangle.

29. The device of claim 1, wherein at least two capture zones are the same shape.

30. The device of claim 1, wherein at least two capture zones are of same size.

31. The device of claim 1, wherein a binding affinity of the analyte binding with the capture agent is higher than a binding affinity of the immobilized conjugate binding with the capture agent.

32. A method for detecting the presence of an analyte in a liquid sample, the method comprising: (i) contacting the liquid sample to the sample receiving zone of the device of any one of claims 1-31, wherein the capture agent comprises a detectable label; and (ii) observing a detectable signal from the detectable label in the capture zones, wherein the detectable signal is inversely proportional to a concentration of the analyte in the sample.

33. The method according to claim 32, further comprising combining the detectable signals from two or more capture zones to provide a processed signal.

34. The method according to claim 33, wherein the detectable signal is provided as a quantified value and combining comprises summing the detectable signals from said two or more capture zones to provide the processed signal.

35. The method according to claim 33, wherein the detectable signal is provided as a quantified value and said combining the detectable signals comprises averaging the detectable signal from said two or more capture zones to provide the processed signal.

36. A device for detecting the presence of a small molecule analyte in a liquid sample, comprising:
a lateral flow matrix which defines a flow path and which comprises in series:
a sample receiving zone; and
one or more capture zones,
wherein each capture zone independently comprises a conjugate immobilized on the lateral flow matrix, and an amount of the immobilized conjugate in a capture zone closer to the sample receiving zone is lower than an amount of the immobilized conjugate in a capture zone further from the sample receiving zone.

37. The device according to claim 36, wherein the conjugate comprises one or more analyte-related molecules conjugated to the lateral flow matrix via a linker and wherein the linker is not a protein, and wherein the conjugate is adapted for orienting the analyte-related molecule for binding with a capture agent capable of binding specifically with the analyte.

38. The device according to claim 36, wherein an amount of the immobilized conjugate in each capture zone is lower than an amount of the immobilized conjugate in each capture zone that is further from the sample receiving zone.
